(19) United States
(12) Patent Application Publication
(10) Pub. No.: US 2025/0302970 A1
(43) Pub. Date: Oct. 2, 2025

(54) USES OF AMPHIPHILES IN IMMUNE CELL THERAPY AND COMPOSITIONS THEREFOR

(71) Applicants: Elicio Therapeutics, Inc., Boston, MA (US); The Trustees of the University of Pennsylvania, Philadelphia, PA (US)

(72) Inventors: Michael BABOKHOV, Boston, MA (US); Peter C. DEMUTH, Boston, MA (US); Zev A. BINDER, Philadelphia, PA (US); Meghan T. LOGUN, Philadelphia, PA (US); Donald M. O'ROURKE, Wynnewood, PA (US); Jesse RODRIGUEZ, Philadelphia, PA (US)

(21) Appl. No.: 18/864,070

(22) PCT Filed: May 10, 2023
(86) PCT No.: PCT/US2023/066827

§ 371 Date: Nov. 8, 2024

Related U.S. Application Data
(60) Provisional application No. 63/340,240, filed on May 10, 2022.

(51) Int. Cl. A61K 47/54; A61P 35/00; C07K 14/71

(52) U.S. Cl. CPC A61K 47/544; A61P 35/00; C07K 14/71

(57) ABSTRACT

The disclosure features amphiphilic ligand conjugates including an EGFR peptide as well as immune cells modified with EGFR receptors. The disclosure also features compositions and methods of using the same, for example, to stimulate proliferation of T cell receptor expressing cells.

### SEQUENCE LISTING

The instant application contains a Sequence Listing which has been submitted electronically in XML format and is hereby incorporated by reference in its entirety. Said XML copy, created on May 10, 2023, is named 51026-053WO2_SL and is 50,974 bytes in size.

### BACKGROUND OF THE INVENTION

Cancer is one of the leading causes of death in the world, with over 14 million new cancer cases diagnosed and over eight million cancer deaths occurring each year. The American Cancer Society estimates 1,762,450 new cases of cancer and 606,880 cancer deaths in the United States in 2019. While several treatments for cancer have been developed, the disease still remains a significant problem.

There thus exists a need for improved treatments for cancer.

### SUMMARY OF THE INVENTION

The invention provides, inter alia, an amphiphilic ligand conjugate, or a pharmaceutically acceptable salt thereof, including a lipid, an EGFR peptide (e.g., one including an amino acid sequence of SEQ ID NO: 1, 2, or 5), and, optionally, a linker. In some embodiments, the EGFR peptide is 7 to 40 amino acids in length. In some embodiments, the EGFR peptide is 10 to 30 amino acids in length. In some embodiments, the EGFR peptide is 15 to 25 amino acids in length. In some embodiments, the EGFR peptide is 15 to 20 amino acids in length. In some embodiments, the EGFR peptide is 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or 40 amino acids in length. In some embodiments, the EGFR peptide is 15 amino acids in length. In some embodiments, the EGFR peptide is 16 amino acids in length. In some embodiments, the EGFR peptide is 17 amino acids in length. In some embodiments, the EGFR peptide is 18 amino acids in length. In some embodiments, the EGFR peptide is 19 amino acids in length. In some embodiments, the EGFR peptide is 20 amino acids in length. In some embodiments, the EGFR peptide is a binding ligand for an EGFR-specific antibody. In some embodiments, the EGFR-specific antibody is antibody 806.

In some embodiments, the EGFR peptide is a cyclic peptide. In some embodiments, the peptide includes an amino acid sequence identical to SEQ ID NO: 1. In some embodiments, the peptide includes an amino acid sequence identical to SEQ ID NO: 2. In some embodiments, the peptide includes an amino acid sequence identical to SEQ ID NO: 5.

In some embodiments, the lipid of the amphiphilic ligand conjugate is a diacyl lipid. In some embodiments, the diacyl lipid of the amphiphilic ligand conjugate includes acyl chains including 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units. In some embodiments, the lipid is 1,2-distearoyl-sn-glycero-3-phosphoethanolamine (DSPE).

In some embodiments, the linker is selected from the group consisting of a hydrophilic polymer, a string of hydrophilic amino acids, a polysaccharide, and an oligonucleotide, or a combination thereof. In some embodiments, the linker includes “N” polyethylene glycol units, wherein N is between 24-50 (e.g., N is 24, 25, 26, 27, 28, 29, 30, 31, 32, 33, 34, 35, 36, 37, 38, 39, 40, 41, 42, 43, 44, 45, 46, 47, 48, 49, and 50). In some embodiments, the linker includes PEG24-amido-PEG24.

In some embodiments, the amphiphilic ligand conjugate is trafficked to a lymph node. In some embodiments, the amphiphilic ligand conjugate is trafficked to an inguinal lymph node or an axillary lymph node. In some embodiments, the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

In another aspect, the disclosure provides a method of stimulating an immune response to a target cell population or target tissue in a subject including administering to the subject (1) an amphiphilic ligand conjugate, including a lipid, an EGFR peptide (e.g., one including an amino acid sequence of SEQ ID NO: 1, 2, or 5) and, optionally, a linker, and (2) an immune cell modified with an EGFR receptor, wherein the EGFR receptor binds the EGFR peptide of the amphiphilic ligand conjugate.

In some embodiments, the method further includes administering an adjuvant to the subject. In some embodiments, the adjuvant is an amphiphilic oligonucleotide conjugate including an immunostimulatory oligonucleotide conjugated to a lipid, with or without a linker.

In some embodiments, the EGFR peptide is a cyclic peptide. In some embodiments, the peptide includes an amino acid sequence identical to SEQ ID NO: 1. In some embodiments, the peptide includes an amino acid sequence identical to SEQ ID NO: 2. In some embodiments, the peptide includes an amino acid sequence identical to SEQ ID NO: 5.

In some embodiments, the EGFR receptor is an EGFRviii receptor.

In some embodiments, the lipid of the amphiphilic ligand conjugate inserts into a cell membrane under physiological conditions, binds albumin under physiological conditions, or both. In some embodiments, the lipid of the amphiphilic ligand conjugate is a diacyl lipid. In some embodiments, the diacyl lipid of the amphiphilic ligand conjugate includes acyl chains including 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units. In some embodiments, the lipid is 1,2-distearoyl-sn-glycero-3-phosphoethanolamine (DSPE).

In some embodiments, the linker is present and is selected from the group consisting of a hydrophilic polymer, a string of hydrophilic amino acids, a polysaccharide, and an oligonucleotide, or a combination thereof. In some embodiments, the linker includes “N” polyethylene glycol units, wherein N is between 24-50 (e.g., N is 24, 25, 26, 27, 28, 29, 30, 31, 32, 33, 34, 35, 36, 37, 38, 39, 40, 41, 42, 43, 44, 45, 46, 47, 48, 49, and 50). In some embodiments, the linker includes PEG24-amido-PEG24.

In some embodiments, the amphiphilic ligand conjugate is trafficked to a lymph node. In some embodiments, the amphiphilic ligand conjugate is trafficked to an inguinal lymph node or an axillary lymph node. In some embodiments, the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

In some embodiments, the immune cell is a T cell, a B cell, a natural killer (NK) cell, a macrophage, a neutrophil, a dendritic cell, a mast cell, an eosinophil, or a basophil. In some embodiments, the immune cell is a T cell.

In some embodiments, the immune response is an anti-tumor immune response. In some embodiments, the target cell population or the target tissue is a tumor cell population or a tumor tissue.

In some embodiments, the method includes reducing or decreasing the size of the tumor tissue or inhibiting growth of the tumor cell population or the tumor tissue in the subject. In some embodiments, the method includes activating the immune cell, expanding the immune cell, and/or increasing proliferation of the immune cell.

In some embodiments, the subject has a disease, a disorder, or a condition associated with expression or elevated expression of the antigen.

In some embodiments, the subject is lymphodepleted prior to the administration of the amphiphilic lipid conjugate. In some embodiments, the lymphodepletion is by sublethal irradiation.

In some embodiments, the subject is administered the amphiphilic ligand conjugate prior to receiving the immune cell modified with an EGFR receptor. In some embodiments, the subject is administered the amphiphilic ligand conjugate after receiving the immune cell modified with an EGFR receptor. In some embodiments, amphiphilic ligand conjugate of and the receiving immune cell modified with an EGFR receptor are administered simultaneously.

In some embodiments, the lipid is conjugated directly or via a linker to the N-terminus of the EGFR peptide.

In some embodiments, the lipid is conjugated directly or via a linker to the C-terminus of the EGFR peptide.

In another aspect, the disclosure provides a kit including (1) an amphiphilic ligand conjugate or pharmaceutically acceptable salt thereof, and (2) an immune cell modified with an EGFR receptor, wherein the EGFR receptor binds the EGFR peptide of the amphiphilic ligand conjugate. In some embodiments, the EGFR receptor is an EGFRviii receptor.

### BRIEF DESCRIPTION OF THE DRAWINGS

FIG. 1 shows a schematic of the mouse cell lines and amphiphilic conjugates that were used in a killing assay experiment which included the B16-F10 cell line expressing firefly luciferase (B16), a B16-F10 cell line expressing a retrovirally transduced EFGRviii receptor (B16-EGFRviii), a B16-F10 cell labeled with amphiphilic conjugate including an EGFR peptide and a lipid at either the N-terminus or C-terminus of the EGFR peptide (B16-AMP-EGFR and EGFR-806-AMP, respectively). A disulfide bond is indicated by the two connected black spheres in the cyclic peptide.

FIG. 2 is a series of graphs showing the transduction efficiency of mouse T cells isolated from B6 mice spleens retrovirally transduced with nothing (UTD), an mCherry control (mCherry), or an EGFR receptor (aEGFR). The mouse T cells expressing EGFRviii receptors at 62% transduction efficiency were used as the positive control.

FIG. 3A-FIG. 3C are a series of graphs showing percent lysis of B16-F10 mice tumor cells expressing firefly luciferase that were co-incubated with an mCherry negative control (mCherry) (FIG. 3A), an EGFRviii receptor (SEQ ID NO: 9) (FIG. 3B), or an EGFR receptor (SEQ ID NO: 8) (FIG. 3C) in triplicate overnight at various effector-to-target (E:T) ratios.

FIG. 4 is a schematic of the human cell lines and amphiphilic conjugates that were used in a killing assay experiment which included the U87-MG cell line expressing firefly luciferase (U87-MG), the Raji cell line expressing firefly luciferase, a Raji cell labeled with amphiphilic conjugate including an EGFR peptide and a lipid at either the N-terminus or C-terminus of the EGFR peptide (Raji-AMP-EGFR and Raji-EGFR-AMP, respectively). A disulfide bond is indicated by the two connected black spheres in the cyclic peptide.

FIG. 5 is a series of graphs showing the transduction efficiency of human T cells isolated from leukopaks retrovirally transduced with nothing (UTD), an mCherry control (mCherry), or a retrovirally transduced with an EGFR receptor (αEGFR).

FIG. 6 is a graph showing percent lysis of U87-MG cells that were co-incubated with a human T cell transduced with an mCherry negative control (mCherry) or U87-MG cells that were co-incubated with a human T cell transduced with an EGFR receptor (αEGFR) in triplicate overnight at various effector-to-target (E:T) ratios.

FIG. 7 is a graph showing the amount of cytokine production following co-culturing U87-MG cells with mCherry (U87: mCherry) or EGFR CAR-expressing human T cells (U87: αEGFR), co-culturing unlabeled Raji cells with mCherry (Raji: mCherry) or EGFR CAR-expressing human T cells (Raji: αEGFR), or co-culturing Raji cells labeled with 400 nmol of AMP-EGFR with either mCherry (Raji-AMP-EGFR: mCherry) or EGFR CAR-expressing human T cells (Raji-AMP-EGFR: αEGFR) overnight at an E: T ratio of 2:1.

### Definitions

Terms used in the claims and specification are defined as set forth below unless otherwise specified.

It must be noted that, as used in the specification and the appended claims, the singular forms “a,” “an,” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide. In some embodiments, the adjuvant is an immunostimulatory oligonucleotide as described herein. In some embodiments, the adjuvant is administered prior to, concurrently, or after administration of an amphiphilic ligand conjugate, or composition comprising the conjugate. In some embodiments, the adjuvant is co-formulated in the same composition as an amphiphilic ligand conjugate.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g., by insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, “amphiphile” or “amphiphilic” refers to a conjugate comprising a hydrophilic head group and a hydrophobic tail, thereby forming an amphiphilic conjugate. In some embodiments, an amphiphile conjugate comprises a peptide and one or more hydrophobic lipid tails, referred to herein as an “amphiphilic ligand conjugate.” In some embodiments, an amphiphile conjugate comprises an EGFR peptide and one or more hydrophobic lipid tails. In some embodiments, the amphiphile conjugate further comprises a polymer (e.g., polyethylene glycol), wherein the polymer is conjugated to the one or more lipids or the peptide.

The term “ameliorating” refers to any therapeutically beneficial result in the treatment of a disease state, e.g., cancer, including prophylaxis, lessening in the severity or progression, remission, or cure thereof.

The term “antibody 806” refers to the antibody MAb 806, which is a mouse lgG2b monoclonal antibody that was raised against human EGFRviii protein as described in Johns et al. Int. J. Cancer: 98, 398-408 (2002).

The term “antigen presenting cell” or “APC” is a cell that displays foreign antigen complexed with MHC on its surface. T cells recognize this complex using T cell receptor (TCR). Examples of APCs include, but are not limited to, dendritic cells (DCs), peripheral blood mononuclear cells (PBMC), monocytes (such as THP-1), B lymphoblastoid cells (such as CIR.A2 and 1518 B-LCL) and monocyte-derived dendritic cells (DCs). Some APCs internalize antigens either by phagocytosis or by receptor-mediated endocytosis.

As used herein, the term “antigenic formulation” or “antigenic composition” or “immunogenic composition” refers to a preparation which, when administered to a vertebrate, especially a mammal, will induce an immune response.

As used herein, the term “cyclic peptide” refers to a polypeptide chain that forms a ring structure through a series of bonds. The bond that connects the polypeptide chain into the ring structure may be between the amino and carboxy ends of the peptide, the carboxy end of the peptide and a side chain of an amino acid residue, or between two side chains of two amino acid residues. In particular embodiments, the cyclic peptide is formed by a disulfide bond between two cysteine residues.

The “intracellular signaling domain” means any oligopeptide or polypeptide domain known to function to transmit a signal causing activation or inhibition of a biological process in a cell, for example, activation of an immune cell such as a T cell or a NK cell. Examples include ILR chain, CD28, and/or CD38.

As used herein, “CG oligodeoxynucleotides (CG ODNs)”, also referred to as “CpG ODNs”, are short single-stranded synthetic DNA molecules that contain a cytosine nucleotide (C) followed by a guanine nucleotide (G). In certain embodiments, the immunostimulatory oligonucleotide is a CG ODN.

As used herein the term “co-stimulatory ligand” includes a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-I (CD80), B7-2 (CD86), PD-L1, PD-L2, 4-1BBL, OX40L, inducible costimulatory ligand (ICOS-L), intercellular adhesion molecule (rCAM), CD30L, CD40, CD70, CD83, HLA-G, MICA, MICE, HVEM, lymphotoxin beta receptor, TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-I, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to, an MHC class I molecule, BTLA, and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A polypeptide or amino acid sequence “derived from” a designated polypeptide or protein or a “polypeptide fragment” refers to the origin of the polypeptide. Preferably, the polypeptide or amino acid sequence which is derived or is a fragment of is from a particular sequence that has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from or that are fragments of another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

As used herein, the term antigen “cross-presentation” refers to presentation of exogenous protein antigens to T cells via MHC class I and class II molecules on APCs.

As used herein, the term “cytotoxic T lymphocyte (CTL) response” refers to an immune response induced by cytotoxic T cells. CTL responses are mediated primarily by CD8+ T cells.

As used herein, the term “effective dose” or “effective dosage” is defined as an amount sufficient to achieve or at least partially achieve the desired effect.

The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “effector cell” or “effector immune cell” refers to a cell involved in an immune response, e.g., in the promotion of an immune effector response. In some embodiments, immune effector cells specifically recognize an antigen. Examples of immune effector cells include, but are not limited to, Natural Killer (NK) cells, B cells, monocytes, macrophages, T cells (e.g., cytotoxic T lymphocytes (CTLs). In some embodiments, the effector cell is a T cell.

As used herein, the term “EGFR peptide” refers to a peptide that includes a segment of the epidermal growth factor receptor (EGFR) (e.g., human EGFR). The EGFR peptide is a short peptide that includes fewer than 30 amino acid residues. In some embodiments, the EGFR peptide includes 18, 19, or 20 amino acid residues. As used herein, the term “immune effector function” or “immune effector response” refers to a function or response of an immune effector cell that promotes an immune response to a target.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes). In particular embodiments, the immune cell is T cell.

As used herein, “immune response” refers to a response made by the immune system of an organism to a substance, which includes but is not limited to foreign or self-proteins. Three general types of “immune response” include mucosal, humoral, and cellular immune responses. For example, the immune response can include the activation, expansion, and/or increased proliferation of an immune cell (e.g., a T cell). An immune response may also include at least one of the following: cytokine production, T cell activation and/or proliferation, granzyme or perforin production, activation of antigen presenting cells or dendritic cells, antibody production, inflammation, developing immunity, developing hypersensitivity to an antigen, the response of antigen-specific lymphocytes to antigen, clearance of an infectious agent, and transplant or graft rejection.

As used herein, an “immunostimulatory oligonucleotide” is an oligonucleotide that can stimulate (e.g., induce or enhance) an immune response.

The terms “inducing an immune response” and “enhancing an immune response” are used interchangeably and refer to the stimulation of an immune response (i.e., either passive or adaptive) to a particular antigen.

The term “induce” as used with respect to inducing complement dependent cytotoxicity (CDC) or antibody-dependent cellular cytotoxicity (ADCC) refer to the stimulation of particular direct cell killing mechanisms.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an amphiphilic ligand conjugate).

The term “in vivo” refers to processes that occur in a living organism.

The term “in vitro” refers to processes that occur outside a living organism, such as in a test tube, flask, or culture plate.

As used herein, the terms “linked,” “operably linked,” “fused,” or “fusion,” are used interchangeably. These terms refer to the joining together of two more elements or components or domains, by an appropriate means including chemical conjugation or recombinant DNA technology. Methods of chemical conjugation (e.g., using heterobifunctional crosslinking agents) are known in the art as are methods of recombinant DNA technology.

The term “lipid” refers to a biomolecule that is soluble in nonpolar solvents and insoluble in water. Lipids are often described as hydrophobic or amphiphilic molecules which allows them to form structures such as vesicles or membranes in aqueous environments. Lipids include fatty acids, glycerolipids, glycerophospholipids, sphingolipids, sterol lipids (including cholesterol), prenol lipids, saccharolipids, and polyketides. In some embodiments, the lipid suitable for the amphiphilic ligand conjugates of the disclosure binds to human serum albumin under physiological conditions. In some embodiments, the lipid suitable for the amphiphilic ligand conjugates of the disclosure inserts into a cell membrane under physiological conditions. In some embodiments, the lipid binds albumin and inserts into a cell membrane under physiological conditions. In some embodiments, the lipid is a diacyl lipid. In some embodiments, the diacyl lipid includes at least 12 carbons. In some embodiments, the diacyl lipid includes 12-30 hydrocarbon units, 14-25 hydrocarbon units, or 16-20 hydrocarbon units. In some embodiments, the diacyl lipid includes 12, 13, 14, 15, 16, 17, 18, 19, 20, 21, 22, 23, 24, 25, 26, 27, 28, 29 or 30 carbons.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mal. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single-and double-stranded regions, hybrid molecules comprising DNA and RNA that can be single-stranded or, more typically, double-stranded or a mixture of single- and double stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms. In some embodiments, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “physiological conditions” refers to the in vivo condition of a subject. In some embodiments, physiological condition refers to a neutral pH (e.g., pH between 6-8).

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “subject” or “mammal” or “patient” includes any human or non-human animal. For example, the methods and compositions of the present invention can be used to treat a subject with a cancer or infection. The term “non-human animal” includes all vertebrates, e.g., mammals and non-mammals, such as non-human primates, sheep, dogs, cats, mice, horses, pigs, cows, chickens, amphibians, reptiles, etc.

The term “sufficient amount” or “amount sufficient to” means an amount sufficient to produce a desired effect, e.g., an amount sufficient to reduce the diameter of a tumor.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}, T_{H}2, T_{H}3, T_{H}17, T_{H}9, and T_{FH} cells, cytotoxic T cells (i.e., To cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. Treg cells or suppressor T cells) and subtypes, including CD4⁺FOXP3⁺ T_{reg} cells, CD4⁺FOXP3⁻ T_{reg} cells, Tr1 cells, Th3 cells, and T_{reg} 17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention.

As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. T cell activation requires at least two signals to become fully activated. The first occurs after engagement of the T cell antigen-specific receptor (TCR) by the antigen-major histocompatibility complex (MHC), and the second by subsequent engagement of co-stimulatory molecules (e.g., CD28). These signals are transmitted to the nucleus and result in clonal expansion of T cells, upregulation of activation markers on the cell surface, differentiation into effector cells, induction of cytotoxicity or cytokine secretion, induction of apoptosis, or a combination thereof.

As used herein, the term “T cell-mediated response” refers to any response mediated by T cells, including, but not limited to, effector T cells (e.g., CD8⁺ cells) and helper T cells (e.g., CD4⁺ cells). T cell mediated responses include, for example, T cell cytotoxicity and proliferation.

The term “T cell cytotoxicity” includes any immune response that is mediated by CDS⁺ T cell activation. Exemplary immune responses include cytokine production, CD8⁺ T cell proliferation, granzyme or perforin production, and clearance of an infectious agent.

As used herein, the term “target-binding domain” of an extracellular domain refers to a polypeptide found on the outside of the cell that is sufficient to facilitate binding to a target. The target-binding domain will specifically bind to its binding partner, i.e., the target. As non-limiting examples, the target-binding domain can include an antigen-binding domain of an antibody, or a ligand, which recognizes and binds with a cognate binding partner protein. In this context, a ligand is a molecule that binds specifically to a portion of a protein and/or receptor. The cognate binding partner of a ligand useful in the methods and compositions described herein can generally be found on the surface of a cell. Ligand: cognate partner binding can result in the alteration of the ligand-bearing receptor, or activate a physiological response, for example, the activation of a signaling pathway. In one embodiment, the ligand can be non-native to the genome. Optionally, the ligand has a conserved function across at least two species. In some embodiments, the ligand is a cancer antigen. In some embodiments, the ligand is a tumor-associated antigen.

A “therapeutic antibody” is an antibody, fragment of an antibody, or construct that is derived from an antibody, and can bind to a cell-surface antigen on a target cell to cause a therapeutic effect. Such antibodies can be chimeric, humanized or fully human antibodies. Methods are known in the art for producing such antibodies. Such antibodies include single chain Fv fragments of antibodies, minibodies and diabodies. Any of the therapeutic antibodies known in the art to be useful for cancer therapy can be used in combination therapy with the compositions described herein. Therapeutic antibodies may be monoclonal antibodies or polyclonal antibodies. In preferred embodiments, the therapeutic antibodies target cancer antigens.

As used herein, “therapeutic protein” refers to any polypeptide, protein, protein variant, fusion protein and/or fragment thereof which may be administered to a subject as a medicament. The term “therapeutically effective amount” is an amount that is effective to ameliorate a symptom of a disease. A therapeutically effective amount can be a “prophylactically effective amount” as prophylaxis can be considered therapy.

The terms “treat,” “treating,” and “treatment,” as used herein, refer to therapeutic or preventative measures described herein. The methods of “treatment” employ administration to a subject, in need of such treatment, an amphiphilic ligand conjugate of the present disclosure, for example, a subject receiving T cell immunotherapy. In some embodiments, an amphiphilic ligand conjugate is administered to a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “tumor-associated antigen” refers to an antigen that is produced in a tumor and can be detected by the immune system to trigger an immune response. Tumor-associated antigens have been identified in many human cancers including lung, skin, hematologic, brain, liver, breast, rectal, bladder, and stomach cancers.

As used herein, “vaccine” refers to a formulation which contains an amphiphilic ligand conjugate and an immune cell (e.g., a T cell) modified with an EGFR receptor (e.g., an EGFR receptor encoded by a nucleic acid sequence of SEQ ID NO: 7, 8, 9, or 10) as described herein, optionally combined with an adjuvant, which is in a form that is capable of being administered to a vertebrate and which induces a protective immune response sufficient to induce immunity to prevent and/or ameliorate a disease or condition (e.g., cancer) and/or to reduce at least one symptom of a disease or condition (e.g., cancer) and/or to enhance the efficacy of an immune cell modified with an EGFR receptor. Typically, the vaccine comprises a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine provokes an immune response including, but not limited to, the inducing a protective immune response to induce immunity to prevent and/or ameliorate a disease or condition (e.g., cancer) and/or to reduce at least one symptom of a disease or condition and/or to enhance the efficacy of immune cells (e.g., a T cells) modified with an EGFR receptor (e.g., an EGFR receptor encoded by a nucleic acid sequence of SEQ ID NO: 7, 8, 9, or 10).

### DETAILED DESCRIPTION

Described herein are amphiphilic conjugates including a lipid and EGFR peptide having an amino acid sequence of ACGADSYEMEEDGVRKCK (SEQ ID NO: 5), ACGADSYEMEEDGVRKCK (AMP-Cys) (SEQ ID NO: 1), or (AMP-Cys) ACGADSYEMEEDGVRKCK (SEQ ID NO: 2), and optionally a linker, where the terminal Cys residue of SEQ ID NO: 1 and the initial Cys residue of SEQ ID NO: 2 is conjugated to the lipid. Further described herein are methods for stimulating an immune response to a target cell population in a subject, where the methods include administering to the subject an amphiphilic lipid conjugates described herein, and an immune cell (e.g., a T cell) modified with an EGFR receptor (e.g., one encoded by the sequence of SEQ ID NO: 7, 8, 9, or 10). Such methods are useful for, e.g., treating a human subject with cancer.

### Amphiphilic Conjugates

The amphiphilic conjugates described herein include a lipid and an EGFR peptide. The EGFR peptide of the amphiphilic lipid conjugate includes the amino acid sequence of SEQ ID NO: 1, SEQ ID NO: 2, or SEQ ID NO: 5. In certain embodiments, amphiphilic conjugates described herein are used with an EGFR receptor (e.g., an EGFR receptor encoded by a nucleic acid sequence of SEQ ID NO: 7, 8, 9, or 10) expressing immune cell therapy. The amphiphilic conjugates may stimulate a specific immune response against a specific target, such as a tumor-associated antigen. In some embodiments, the amphiphilic conjugate induces activation, expansion, or proliferation of an immune cell (e.g., a T cell) expressing an EGFR receptor (e.g., one encoded by the sequence of SEQ ID NO: 7, 8, 9, or 10) in vivo. In some embodiments, the amphiphilic conjugate includes a ligand, and is referred to herein as an “amphiphilic ligand conjugate.”

The structure of an amphiphilic ligand conjugate as described herein includes a lipophilic moiety, or “lipid tail”, (e.g., DSPE) covalently linked, optionally via a linker (e.g., PEG-2000), to one or more cargos. The amphiphilic ligand conjugate cargo can include an EGFR peptide having the amino acid sequence of any one of SEQ ID NOs: 1 or 2. The lipophilic moiety may be conjugated to the peptide cargo at the N-terminus or C-terminus of the peptide.

Upon administration, without being bound by theory, the amphiphilic ligand conjugate is thought to be delivered to lymph nodes where the lipid tail portion is inserted into the membrane of antigen presenting cells (APCs), resulting in the decoration of the APC with ligands. The embedded ligands function as specific targets for an engineered receptor (i.e., an EGFR receptor (e.g., an EGFR receptor encoded by a nucleic acid sequence of SEQ ID NO: 7, 8, 9, or 10)) expressed on the surface of prior, subsequent, or co-administered immune cells (e.g., T cells) expressing said receptor, resulting in the recruitment of the immune cells (e.g., a T cells) to the ligand-decorated APCs. Interaction of the engineered receptor with the embedded ligand provides a stimulatory signal through the engineered receptor while the APC additionally presents other naturally occurring co-stimulatory signals, resulting in optimal immune cell (e.g., T cell) activation, prolonged survival, and efficient memory formation.

Amphiphilic ligand conjugates can be generated using methods known in the art, such as those described in US 2013/0295129, which is hereby incorporated by reference in its entirety. For example, N-terminal cysteine modified peptides can be dissolved in DMF (dimethylformamide) and mixed with 2 equivalents Maleimide-PEG2000-DSPE (Laysan Bio, Inc.), and agitating the mixture at room temperature for 24 hours. Bioconjugation can be assessed by HPLC analysis.

### Amphiphilic Ligand Conjugate Cargos

The peptide can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids. In some embodiments, a peptide can be greater than 50 amino acids. In some embodiments, the peptide can be >100 amino acids. For example, the peptide may include between 18 and 20 amino acids (e.g., 18, 19, or 20 amino acids).

In some embodiments, the peptide is an EGFR peptide, and the EGFR peptide is 7 to 40 amino acids in length. In some embodiments, the EGFR peptide is 10 to 30 amino acids in length. In some embodiments, the EGFR peptide is 15 to 25 amino acids in length. In some embodiments, the EGFR peptide is 15 to 20 amino acids in length. In some embodiments, the EGFR peptide is 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, or 40 amino acids in length. In some embodiments, the EGFR peptide is 15 amino acids in length. In some embodiments, the EGFR peptide is 16 amino acids in length. In some embodiments, the EGFR peptide is 17 amino acids in length. In some embodiments, the EGFR peptide is 18 amino acids in length. In some embodiments, the EGFR peptide is 19 amino acids in length. In some embodiments, the EGFR peptide is 20 amino acids in length.

The protein or polypeptide can be any protein or peptide that can induce or increase the ability of the immune system to develop antibodies and T cell responses to the protein or peptide.

A T cell receptor ligand of an amphiphilic ligand conjugate described herein can be an EGFR peptide. The EGFR peptide may be a fragment of SEQ ID NO: 11.

In some embodiments, the peptide includes the amino acid sequence of ACGADSYEMEEDGVRKCK (SEQ ID NO: 5). The peptide may include an amino acid sequence of ACGADSYEMEEDGVRKCK (AMP-Cys), SEQ ID NO: 1, wherein the lipid is conjugated to the C-terminus of the peptide, and where an internal disulfide bridge forms between the other two Cys residues, corresponding to Cys287 and Cys302 (see, Johns et al. J. Biol. Chem. 279:29. 30375-30384 2004). In some embodiments, the peptide has an amino acid sequence of (AMP-Cys) ACGADSYEMEEDGVRKCK (SEQ ID NO: 2), where the lipid is conjugated to the N-terminus, and where an internal disulfide bridge forms between the other two Cys residues, corresponding to Cys287 and Cys302 (see, Johns et al. J. Biol. Chem. 279:29. 30375-30384 2004).

The protein/peptide can be linear, branched, or cyclic. In some embodiments, the peptide is cyclic. In some embodiments, the cyclic peptide is formed by way of a disulfide bond. For example, the cyclic peptide may be formed by way of a disulfide bond between the first Cys residue at the N-terminus and the last Cys residue at the C-terminus. The peptide can include D amino acids, L amino acids, or a combination thereof.

The peptide or protein can be conjugated to the polar block or lipid at the N-terminus or the C-terminus of the peptide or protein.

In some embodiments, the methods and compositions of the disclosure are used in combination with Kymriah™ (tisagenlecleucel; Novartis) suspension for intravenous infusion, formerly CTL019.

Suitable antigens are known in the art and are available from commercial, government, and scientific sources. The antigens may be purified, or partially purified polypeptides derived from tumors. The antigens can be recombinant polypeptides produced by expressing DNA encoding the polypeptide antigen in a heterologous expression system. The DNA may be in the form of vector DNA such as plasmid DNA.

In certain embodiments, antigens may be provided as single antigens or may be provided in combination. Antigens may also be provided as complex mixtures of polypeptides or nucleic acids.

### Lipid Conjugates

In certain embodiments, a lipid conjugate (e.g., an amphiphilic ligand conjugate), as described in US 2013/0295129, herein incorporated by reference in its entirety, is used in the methods disclosed herein. A lipid conjugate includes an albumin-binding lipid and a cargo to efficiently target the cargo to lymph nodes in vivo. Lipid conjugates bind to endogenous albumin, which targets them to lymphatics and draining lymph nodes where they accumulate due to the filtering of albumin by antigen presenting cells. The lipid conjugate may include an antigenic peptide (e.g., an EGFR peptide e.g., SEQ ID NOs: 1 or 2) or molecular adjuvant and thereby induces or enhances a robust immune response. In some embodiments, the lipid conjugate includes a T cell receptor ligand, and thereby can induce or enhance expansion, proliferation, and/or activation of immune cells (e.g., T cells) expressing a T cell receptor (e.g., an EGFR receptor, e.g., an EGFR receptor encoded by a nucleic acid sequence of SEQ ID NO: 7, 8, 9, or 10)).

Lymph node-targeting conjugates typically include three domains: a highly lipophilic, albumin-binding domain (e.g., an albumin-binding lipid), a cargo such as a peptide (e.g., an EGFR peptide) or molecular adjuvant, and a polar block linker, which promotes solubility of the conjugate and reduces the ability of the lipid to insert into cellular plasma membranes. Accordingly, in certain embodiments, the general structure of the conjugate is L-P-C, where “L” is an albumin-binding lipid, “P” is a polar block, and “C” is a cargo such as a peptide (e.g., an EGFR peptide) or a molecular adjuvant. In some embodiments, the cargo itself can also serve as the polar block domain, and a separate polar block domain is not required. Therefore, in certain embodiments the conjugate has only two domains: an albumin-binding lipid and a cargo, e.g., a peptide.

In some embodiments, the cargo of the conjugate is a peptide (e.g., an EGFR peptide), such as in an amphiphilic ligand conjugate. In other embodiments, the peptide is an antigenic peptide. In further embodiments, the peptide is a tumor associated antigenic peptide. In yet other embodiments, the peptide is a T cell receptor target, e.g., an epitope. In some embodiments, the amphiphilic ligand conjugate is administered or formulated with an adjuvant, wherein the adjuvant is an amphiphilic conjugate including a molecular adjuvant such as an immunostimulatory oligonucleotide.

Optionally, the amphiphilic ligand conjugate is in the form of a pharmaceutically acceptable salt. The term “pharmaceutically acceptable salt,” as used herein, means any pharmaceutically acceptable salt of a conjugate, oligonucleotide, or peptide disclosed herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. References to conjugates (e.g., amphiphilic ligand conjugates), oligonucleotides, or peptides in the claims are to be interpreted to optionally include pharmaceutically acceptable salts thereof.

### Lipids

The amphiphilic ligand conjugates typically include a hydrophobic lipid. The lipid can be linear, branched, or cyclic. In certain embodiments, the activity relies, in part, on the ability of the conjugate to insert itself into a cell membrane. Therefore, lymph node-targeted conjugates typically include a lipid that undergo membrane insertion under physiological conditions. Lipids suitable for membrane insertion can be selected based on the ability of the lipid or a lipid conjugate including the lipid to bind to interact with a cell membrane. Suitable methods for testing the membrane insertion of the lipid or lipid conjugate are known in the art.

Examples of preferred lipids for use in lymph node targeting lipid conjugates include, but are not limited to, fatty acids with aliphatic tails of 3-30 carbons including, but not limited to, linear unsaturated and saturated fatty acids, branched saturated and unsaturated fatty acids, and fatty acids derivatives, such as fatty acid esters, fatty acid amides, and fatty acid thioesters, diacyl lipids, cholesterol, cholesterol derivatives, and steroid acids such as bile acids, Lipid A or combinations thereof.

In certain embodiments, the lipid is a diacyl lipid or two-tailed lipid. In some embodiments, the tails in the diacyl lipid contain from about 12 to about 30 carbons (e.g., 13 14, 15, 16, 17, 18, 19, 20, 21, 22, 23, 24, 25, 26, 27, 28, or 29). In some embodiments the tails in the diacyl lipid contain about 14 to about 25 carbons (e.g., 15, 16, 17, 18, 19, 20, 21, 22, 23, or 24). In some embodiments, the tails of the diacyl lipid contain from about 16 to about 20 carbons (e.g., 17, 18, or 19). In some embodiments, the diacyl lipid comprises 12, 13, 14, 15, 16, 17, 18, 19, 20, 21, 22, 23, 24, 25, 26, 27, 28, 29 or 30 carbons.

The carbon tails of the diacyl lipid can be saturated, unsaturated, or combinations thereof. The tails can be coupled to the head group via ester bond linkages, amide bond linkages, thioester bond linkages, or combinations thereof. In a particular embodiment, the diacyl lipids are phosphate lipids, glycolipids, sphingolipids, or combinations thereof.

Preferably, membrane-inserting conjugates include a lipid that is por fewer carbon units in length, as it is believed that increasing the number of lipid units can reduce insertion of the lipid into plasma membrane of cells, allowing the lipid conjugate to remain free to bind albumin and traffic to the lymph node.

### Molecular Adjuvants

In certain embodiments, amphiphilic oligonucleotide conjugates are used with the amphiphilic ligand conjugate. The oligonucleotide conjugates typically contain an immunostimulatory oligonucleotide.

In certain embodiments, the immunostimulatory oligonucleotide can serve as a ligand for pattern recognition receptors (PRRs). Examples of PRRs include the Toll-like family of signaling molecules that play a role in the initiation of innate immune responses and also influence the later and more antigen specific adaptive immune responses. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76 (5): 2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In certain embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, AM, Advanced drug delivery reviews 61 (3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94 (20): 10833-7 (1997); Dalpke, AH, Immunology 106 (1): 102-12 (2002); Hartmann, G, J of Immun. 164 (3): 1617-2 (2000), each of which is incorporated herein by reference).

According to some embodiments, a lipophilic-CpG oligonucleotide conjugate is used to enhance an immune response to an antigen. An exemplary lipophilic-CpG oligonucleotide conjugate includes the sequence 5′-TCGTCGTTTTGTCGTTTTGTCGTT-3′ (SEQ ID NO:12). The CpG oligonucleotide sequence is linked, at its 5′ end, to a lipid, such as the following, where the nucleotide linkage is as shown below:

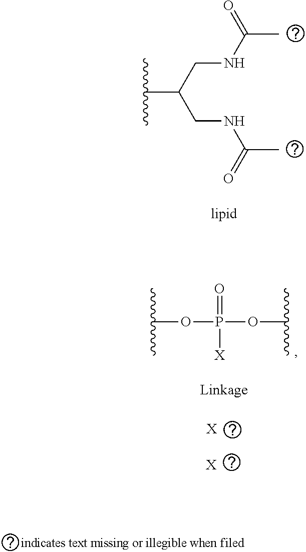

or a salt thereof, wherein X is O or S. Preferably, X is S. The CpG oligonucleotide may be directly bonded to the lipid. Alternatively, the CpG oligonucleotide may be linked to the lipid through a linker, such as GG. In the exemplary CpG oligonucleotide, all internucleoside groups are phosphorothioates (e.g., all internucleoside groups in the compound may be phosphorothioates).

Another exemplary lipophilic-CpG oligonucleotide is represented by the following, wherein “L” is a lipophilic compound, such as diacyl lipid, “Gₙ” is a guanine repeat linker and “n” represents 1, 2, 3, 4, or 5.

(SEQ ID NO: 6)
5′-L-GₙTCCATGACGTTCCTGACGTT-3′

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDA5), which are best known as RNA-sensing receptors in the cytosol. Therefore, in certain embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5 (1): 87-93 (2011), incorporated herein by reference.

In certain embodiments, the oligonucleotide cargo includes two or more immunostimulatory sequences.

The oligonucleotide can be between 2-100 nucleotide bases in length, including for example, 5 nucleotide bases in length, 10 nucleotide bases in length, 15 nucleotide bases in length, 20 nucleotide bases in length, 25 nucleotide bases in length, 30 nucleotide bases in length, 35 nucleotide bases in length, 40 nucleotide bases in length, 45 nucleotide bases in length, 50 nucleotide bases in length, 60 nucleotide bases in length, 70 nucleotide bases in length, 80 nucleotide bases in length, 90 nucleotide bases in length, 95 nucleotide bases in length, 98 nucleotide bases in length, 100 nucleotide bases in length or more.

The 3′ end or the 5′ end of the oligonucleotides can be conjugated to the polar block or the lipid. In certain embodiments the 5′ end of the oligonucleotide is linked to the polar block or the lipid.

The oligonucleotides can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds. In certain embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In certain embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In certain embodiments, the analogs have a substantially uncharged, phosphorus containing backbone.

### Polar Block/Linker

For the conjugate to be trafficked efficiently to the lymph node, the conjugate should remain soluble at the injection site. Therefore, a polar block linker can be included between the cargo and the lipid to increase solubility of the conjugate. The polar block can also reduce or prevent the ability of cargo, such as a peptide, from non-specifically associating with extracellular matrix proteins at the site of administration. The polar block increases the solubility of the conjugate without preventing its ability to bind to albumin. It is believed that this combination of characteristics allows the conjugate to bind to albumin present in the serum or interstitial fluid and remain in circulation until the albumin is trafficked to, and retained in a lymph node.

The length and composition of the polar block can be adjusted based on the lipid and cargo selected.

A polar block can be used as part of any of lipid conjugates suitable for use in the methods disclosed herein, for example, amphiphilic oligonucleotide conjugates and amphiphilic ligand conjugates, which reduce cell membrane insertion/preferential portioning on albumin. Suitable polar blocks include, but are not limited to, oligonucleotides such as those discussed above, a hydrophilic polymer including but not limited to poly(ethylene glycol) (MW: 500 Da to 20,000 Da), polyacrylamide (MW: 500 Da to 20,000 Da), polyacrylic acid; a string of hydrophilic amino acids such as serine, threonine, cysteine, tyrosine, asparagine, glutamine, aspartic acid, glutamic acid, lysine, arginine, histidine, or combinations thereof polysaccharides, including but not limited to, dextran (MW: 1,000 Da to 2,000,000 Da), or combinations thereof.

The hydrophobic lipid and the linker/cargo are covalently linked. The covalent bond may be a non-cleavable linkage or a cleavable linkage. The non-cleavable linkage can include an amide bond or phosphate bond, and the cleavable linkage can include a disulfide bond, acid-cleavable linkage, ester bond, anhydride bond, biodegradable bond, or enzyme-cleavable linkage.

### Ethylene Glycol Linkers

In certain embodiments, the polar block is one or more ethylene glycol (EG) units, more preferably two or more EG units (i.e., polyethylene glycol (PEG)). For example, in certain embodiments, a lipid conjugate includes a protein or peptide (e.g., peptide antigen) and a hydrophobic lipid linked by a polyethylene glycol (PEG) molecule or a derivative or analog thereof.

In certain embodiments, protein conjugates suitable for use in the methods disclosed herein contain protein antigen linked to PEG which is in turn linked to a hydrophobic lipid, or lipid-Gn-ON conjugates, either covalently or via formation of protein-oligo conjugates that hybridize to oligo micelles. The precise number of EG units depends on the lipid and the cargo, however, typically, a polar block can have between about 1 and about 100, between about 20 and about 80, between about 30 and about 70, or between about 40 and about 60 EG units. In certain embodiments, the polar block has between about 45 and 55 EG, units. For example, in certain embodiments, the polar block has 48 EG units. In some embodiments, the EG units are consecutive (e.g., 24 consecutive EG units).

### Egfr Receptor

In some aspects, the disclosure provides compositions and methods to be used or performed in in conjunction with immune cells (e.g., T cells) modified with an EGFR receptor (e.g., an EGFR receptor encoded by a nucleic acid sequence of SEQ ID NO: 7, 8, 9, or 10). Methods described herein include administering to a subject a composition including an amphiphilic ligand conjugate described herein and an immune cell (e.g., a T cell) modified with an EGFR receptor (e.g., an EGFR receptor encoded by a nucleic acid sequence of SEQ ID NO: 7, 8, 9, or 10). In some embodiments, the EGFR receptor binds the peptide of the amphiphilic ligand conjugate. Antigenic peptides bound to MHC molecules are presented to T cells by APC(s). Recognition and engagement of such peptide-MHC complex (pMHC) by the TCR, a molecule found on the surface of T cells, results in T cell activation and response. The EGFR receptor is a heterodimer composed of two different protein chains. In most T cells (about 95%), these two protein chains are alpha (α) and beta (β) chains. However, in a small percentage of T cells (about 5%), these two protein chains are gamma and delta (γ/o) chains. The ratio of TCRs comprised of α/β chains versus γ/δ chains may change during a diseased state (e.g., in cancer (e.g., in a tumor), infectious disease, inflammatory disease or autoimmune disease). Engagement of the TCR with pMHC activates a T cell through a series of biochemical events mediated by associated enzymes, co-receptors, specialized adaptor molecules, and activated or released transcription factors.

Each of the two chains of a TCR contains multiple copies of gene segments-a variable ‘V’ gene segment, a diversity ‘D’ gene segment, and a joining ‘J’ gene segment. The TCR alpha chain is generated by recombination of V and J segments, while the beta chain is generated by recombination of V, D, and J segments. Similarly, generation of the TCR gamma chain involves recombination of V and J gene segments, while generation of the TCR delta chain occurs by recombination of V, D, and J gene segments. The intersection of these specific regions (V and J for the alpha or gamma chain, or V, D and J for the beta or delta chain) corresponds to the CDR3 region that is important for antigen-MHC recognition. Complementarity determining regions (e.g., CDR1, CDR2, and CDR3), or hypervariable regions, are sequences in the variable domains of antigen receptors that can complement an antigen.

CD3 is a T cell co-receptor that facilitates T lymphocyte activation when simultaneously engaged with the appropriate co-stimulation (e.g., binding of a co-stimulatory molecule). A CD3 complex consists of 4 distinct chains; mammalian CD3 consists of a CD3γ chain, a CD30 chain, and two CD38 chains. These chains associate with a T cell receptor (TCR) and CD32 to generate an activation signal in T lymphocytes. A complete TCR complex includes a TCR, CD34, and the complete CD3 complex.

Any immune cell may be modified with an EGFR receptor. For example, the immune cell modified with an EGFR receptor described herein may be a T cell, a B cell, a natural killer (NK) cell, a macrophage, a neutrophil, a dendritic cell, a mast cell, an eosinophil, or a basophil. In some embodiments, the immune cell modified with an EGFR receptor is a T cell.

In particular embodiments, the immune cell (e.g., a T cell) is modified with an EGFRviii receptor. In some embodiments, the immune cell (e.g., T cell) is modified with a human EGFR receptor. In some embodiments, the immune cell (e.g., T cell) is modified with an EGFR receptor encoded by a nucleic acid sequence of SEQ ID NO: 7. In some embodiments, the immune cell (e.g., T cell) is modified with an EGFR receptor encoded by a nucleic acid sequence of SEQ ID NO: 8. In some embodiments, the immune cell (e.g., T cell) is modified with an EGFR receptor encoded by a nucleic acid sequence of SEQ ID NO: 9. In some embodiments, the immune cell (e.g., T cell) is modified with an EGFR receptor encoded by a nucleic acid sequence of SEQ ID NO: 10.

### Modified EGFR Receptor Immune Cells

Engineered immune cell therapy is used to generate immune cells modified with receptors that are capable of recognizing a tumor in a subject. A receptor may recognize a tumor by the antigen present on the major histocompatiblility complex (MHC) the tumor cell surface. In some embodiments, immune cells (e.g., T cells, B cells, NK cells, neutrophils, eosinophils, basophils, and granulocytes) are modified to express a TCR. For example, in particular embodiments, an immune cell (e.g., a T cell) may be modified with an EGFR receptor (e.g., an EGFR receptor encoded by a sequence of SEQ ID NO: 7, 8, 9, or 10) that recognizes and bind an EGFR peptide that may be associated with a tumor. In some embodiments, the modified receptor may be used to activate and expand the immune cell (e.g., T cell) and/or increase proliferation of the immune cell (e.g., T cell). In some embodiments, activating and/or expanding the immune cell (e.g., the T cell) may be done in vitro. In some embodiments, activating and expanding the immune cell (e.g., T cell) may decrease the size of the tumor tissue or inhibit growth of the tumor cell population or tumor tissue in the subject.

Immune cells (e.g., T cells) modified with an EGFR receptor (e.g., an EGFR receptor encoded by a sequence of SEQ ID NO: 7, 8, 9, or 10) may display desired specificities and enhanced functionalities. T cells can be genetically modified to express an EGFR receptor with altered specificity. The T cell may be modified with an EGFR receptor capable of recognizing an EGFR peptide a that is associated with a tumor. For example, in some embodiments, T cells are modified to express a modified an EFGR receptor, where the EGFR receptor binds the EGFR peptide of the amphiphilic ligand conjugate (e.g., SEQ ID NO: 1 or 2). In some embodiments, binding of the EGFR peptide of the amphiphilic ligand conjugate allows for the activation and expansion of T cells directed towards a specific tumor.

An immune cell (e.g., T cell) may be modified with an EGFR receptor by introducing a recombinant nucleic acid encoding an EGFR receptor (e.g., an EGFR receptor encoded by a sequence of SEQ ID NO: 7, 8, 9, or 10) into a patient-derived T cell to generate an EGFR receptor modified immune cell (e.g., T cell). The modified T cell may then be administered back to the subject, for example, after being activated in vitro. In some embodiments, T cells not derived from the subject are genetically modified with an EGFR receptor (e.g., an EGFR receptor encoded by a sequence of SEQ ID NO: 7, 8, 9, or 10). For example, in some embodiments, T cells are allogeneic cells that have been engineered to be used as an “off the shelf” adoptive cell therapy.

A variety of different methods known in the art can be used to introduce any of the nucleic acids or expression vectors disclosed herein into a T cell. Nonlimiting examples of methods for introducing nucleic acid into an immune cell (e.g., T cell) include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection. Furthermore, the CRISPR/Cas9 genome editing technology known in the art can be used to introduce nucleic acids into T cells.

### Immunogenic Compositions

The conjugates suitable for use in the methods disclosed herein can be used in immunogenic compositions or as components in vaccines. Typically, immunogenic compositions disclosed herein include an amphiphilic lipid conjugate including a lipid, an EGFR peptide, optionally a linker. The immunogenic compositions disclosed herein may also include an immune cell (e.g., a T cell) modified with an EGFR receptor (e.g., an EGFR receptor encoded by a sequence of SEQ ID NO: 7, 8, 9, or 10), where the EGFR receptor binds the peptide of the amphiphilic ligand conjugate. The administration to a subject of both an amphiphilic lipid conjugate and an immune cell (e.g., a T cell) modified with an EGFR receptor (e.g., an EGFR receptor encoded by a sequence of SEQ ID NO: 7, 8, 9, or 10) is an example of a vaccine. When administered to a subject in combination, the amphiphilic lipid conjugate and the immune cell (e.g., the T cell) modified with an EGFR receptor (e.g., an EGFR receptor encoded by a sequence of SEQ ID NO: 7, 8, 9, or 10) can be administered in separate pharmaceutical compositions, or they can be administered together in the same pharmaceutical composition. Additionally, the vaccine may include an adjuvant. The adjuvant may be administered in the same pharmaceutical composition as the amphiphilic lipid conjugate and/or the immune cell (e.g., the T cell) modified with an EGFR receptor (e.g., an EGFR receptor encoded by a sequence of SEQ ID NO: 7, 8, 9, or 10), or the adjuvant may be administered in a separate pharmaceutical composition.

### An Immunogenic Composition Suitable for Use in the Methods Disclosed Herein

An immunogenic composition suitable for use in the methods disclosed herein can include the combination of a composition including an amphiphilic ligand conjugate and a composition including an immune cell (e.g., a T cell) modified with an EGFR receptor (e.g., an EGFR receptor encoded by a sequence of SEQ ID NO: 7, 8, 9, or 10). These compositions can be combined into one composition and can be administered alone, or in combination with an adjuvant. In some embodiments, the adjuvant is an amphiphilic oligonucleotide conjugate including an immunostimulatory oligonucleotide, as described supra.

The adjuvant may be, without limitation, alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly [di(carboxylatophenoxy) phosphazene] (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include, without limitation, double-stranded RNA. Adjuvants that act through TLR4 include, without limitation, derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). Adjuvants that act through TLR5 include, without limitation, flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG (Bacillus Colmette-Guérin); mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g., lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-gamma), macrophage colony stimulating factor, and tumor necrosis factor.

### Methods of Making Polypeptides

In some embodiments, the EGFR peptides including the amino acid sequence of SEQ ID NO: 1 or 2 for use in the amphiphilic conjugates are made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The methods of making polypeptides also include a vector capable of expressing the peptides in an appropriate host. The vector includes the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be suitable for use in the methods disclosed herein. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety, and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85:2149; Davis et al. (1985), Biochem. Intl. 10:394-414; Stewart and Young (1969), Solid Phase Peptide Synthesis; U.S. Pat. No. 3,941,763; Finn et al. (1976), The Proteins (3rd ed.) 2:105-253; and Erickson et al. (1976), The Proteins (3rd ed.) 2:257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods of molecule expression/synthesis are generally known in the art to one of ordinary skill.

The nucleic acid molecules of any one of SEQ ID NOs: 1-5 described above can be contained within a vector that is capable of directing their expression in, for example, a cell that has been transduced with the vector. Accordingly, in addition to polypeptide mutants, expression vectors containing a nucleic acid molecule encoding a mutant and cells transfected with these vectors are among the certain embodiments.

Vectors suitable for use include T7-based vectors for use in bacteria (see, for example, Rosenberg et al., Gene 56:125, 1987), the pMSXND expression vector for use in mammalian cells (Lee and Nathans, J. Biol. Chem. 263:3521, 1988), and baculovirus-derived vectors (for example the expression vector pBacPAKS from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 (Geneticin) resistance to cells in which it is expressed, and thus permits phenotypic selection of the transfected cells. Those of skill in the art can readily determine whether a given regulatory element or selectable marker is suitable for use in a particular experimental context.

Viral vectors that are suitable for use include, for example, retroviral, adenoviral, and adeno-associated vectors, herpes virus, simian virus 40 (SV 40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a polypeptide mutant are also suitable for use. A cell is a transfected cell, i.e., a cell into which a nucleic acid molecule, for example a nucleic acid molecule encoding a mutant polypeptide, has been introduced by means of recombinant DNA techniques. The progeny of such a cell is also considered suitable for use in the methods disclosed herein.

The precise components of the expression system are not critical. For example, a polypeptide mutant can be produced in a prokaryotic host, such as the bacterium E. coli, or in a eukaryotic host, such as an insect cell (e.g., an Sf21 cell), or mammalian cells (e.g., COS cells, NIH 3T3 cells, or Hela cells). These cells are available from many sources, including the American Type Culture Collection (Manassas, Va.). In selecting an expression system, it matters only that the components are compatible with one another. Artisans of ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., conjugated to a lipid, as described herein.

### Pharmaceutical Composition and Modes of Administration

In some embodiments, an amphiphilic ligand conjugate including an amino acid sequence of SEQ ID NO: 1 or 2 may be administered as a pharmaceutical composition to a subject. In some embodiments, an amphiphilic ligand conjugate and an immune cell (e.g., a T cell) modified to express an EGFR receptor (e.g., an EGFR receptor encoded by a sequence of SEQ ID NO: 7, 8, 9, or 10) are administered together (simultaneously or sequentially). In some embodiments, an amphiphilic ligand conjugate and an immune cell (e.g., a T cell) modified to express a modified EGFR receptor (e.g., an EGFR receptor encoded by a sequence of SEQ ID NO: 7, 8, 9, or 10) are administered together (simultaneously or sequentially). In some embodiments, the amphiphilic ligand conjugate including a lipid, and optionally a linker is administered to subject. In some embodiments, the amphiphilic ligand conjugate including a lipid is administered to the subject without an immune cell (e.g., a T cell) modified to express an EGFR receptor (e.g., an EGFR receptor encoded by a sequence of SEQ ID NO: 7, 8, 9, or 10). In some embodiments, an amphiphilic ligand conjugate and an adjuvant (e.g., an amphiphilic oligonucleotide conjugate) are administered together (simultaneously or sequentially). In some embodiments, an amphiphilic ligand conjugate, an adjuvant (e.g., an amphiphilic oligonucleotide conjugate), and an immune cell (e.g., a T cell) modified to express an EGFR receptor (e.g., an EGFR receptor encoded by a sequence of SEQ ID NO: 7, 8, 9, or 10) are administered together (simultaneously or sequentially). In some embodiments, an amphiphilic ligand conjugate including a lipid and a peptide having the amino acid sequence of SEQ ID NO: 1 or 2,, and optionally a linker, and an adjuvant (e.g., an amphiphilic oligonucleotide conjugate) are administered together (simultaneously or sequentially). In some embodiments, an amphiphilic ligand conjugate and an immune cell (e.g., a T cell) modified to express an EGFR receptor (e.g., an EGFR receptor encoded by a sequence of SEQ ID NO: 7, 8, 9, or 10) are administered separately. In some embodiments, an amphiphilic ligand conjugate and an adjuvant (e.g., an amphiphilic oligonucleotide conjugate) are administered separately. In some embodiments, an amphiphilic ligand conjugate, an adjuvant (e.g., an amphiphilic oligonucleotide conjugate) and an immune cell (e.g., a T cell) modified to express an EGFR receptor (e.g., an EGFR receptor encoded by a sequence of SEQ ID NO: 7, 8, 9, or 10) are administered separately.

In some embodiments, the disclosure provides for a pharmaceutical composition including an amphiphilic ligand conjugate, having a peptide with an amino acid sequence of SEQ ID NO: 1 or 2, with a pharmaceutically acceptable diluent, carrier, solubilizer, emulsifier, preservative and/or adjuvant. In some embodiments, the adjuvant is an amphiphilic oligonucleotide conjugate.

In some embodiments, acceptable formulation materials preferably are nontoxic to recipients at the dosages and concentrations employed. In certain embodiments, the formulation material(s) are for subcutaneous (s.c.) and/or intravenous (i.v.) administration. In certain embodiments the formulation materials are of intramuscular or transmucosal administration. In certain embodiments, transmucosal administration is nasal administration. In certain embodiments, transmucosal administration is buccal administration. In some embodiments, the pharmaceutical composition can contain formulation materials for modifying, maintaining, or preserving, for example, the pH, osmolality, viscosity, clarity, color, isotonicity, odor, sterility, stability, rate of dissolution or release, adsorption or penetration of the composition. In some embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80 (polyoxyethylene (20) sorbitan monooleate), Triton X-100 (t-Octylphenoxypolyethoxyethanol), tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation includes PBS; 20 mM NaOAc, pH 5.2, 50 mM NaCl; and/or 10 mM NaOAc, pH 5.2, 9% Sucrose. In some embodiments, the optimal pharmaceutical composition will be determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the amphiphilic conjugate.

In some embodiments, the primary vehicle or carrier in a pharmaceutical composition can be either aqueous or non-aqueous in nature. For example, in some embodiments, a suitable vehicle or carrier can be water for injection, physiological saline solution or artificial cerebrospinal fluid, possibly supplemented with other materials common in compositions for parenteral administration. In some embodiments, the saline includes isotonic phosphate-buffered saline. In certain embodiments, neutral buffered saline or saline mixed with serum albumin are further exemplary vehicles. In some embodiments, pharmaceutical compositions include Tris buffer of about pH 7.0-8.5, or acetate buffer of about pH 4.0-5.5, which can further include sorbitol or a suitable substitute therefor. In some embodiments, a composition including an amphiphilic conjugate can be prepared for storage by mixing the selected composition having the desired degree of purity with optional formulation agents (Remington's Pharmaceutical Sciences, supra) in the form of a lyophilized cake or an aqueous solution. Further, in some embodiments, a composition including an amphiphilic conjugate, can be formulated as a lyophilizate using appropriate excipients such as sucrose.

In some embodiments, the pharmaceutical composition can be selected for parenteral delivery. In some embodiments, the compositions can be selected for inhalation or for delivery through the digestive tract, such as orally. The preparation of such pharmaceutically acceptable compositions is within the ability of one skilled in the art.

In some embodiments, the formulation components are present in concentrations that are acceptable to the site of administration. In some embodiments, buffers are used to maintain the composition at physiological pH or at a slightly lower pH, typically within a pH range of from about 5 to about 8.

In some embodiments, when parenteral administration is contemplated, a therapeutic composition can be in the form of a pyrogen-free, parenterally acceptable aqueous solution including an amphiphilic conjugate, in a pharmaceutically acceptable vehicle. In some embodiments, a vehicle for parenteral injection is sterile distilled water in which an amphiphilic conjugate is formulated as a sterile, isotonic solution, properly preserved. In some embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some embodiments, hyaluronic acid can also be used, and can have the effect of promoting sustained duration in circulation. In some embodiments, implantable drug delivery devices can be used to introduce the desired molecule.

In some embodiments, a pharmaceutical composition can be formulated for inhalation. In some embodiments, an amphiphilic conjugate can be formulated as a dry powder for inhalation. In some embodiments, an inhalation solution including an amphiphilic conjugate can be formulated with a propellant for aerosol delivery. In some embodiments, solutions can be nebulized. Pulmonary administration is further described in PCT Publication No. WO/1994/020069, which describes pulmonary delivery of chemically modified proteins.

In some embodiments, it is contemplated that formulations can be administered orally. In some embodiments, an amphiphilic conjugate that is administered in this fashion can be formulated with or without those carriers customarily used in the compounding of solid dosage forms such as tablets and capsules. In some embodiments, a capsule can be designed to release the active portion of the formulation at the point in the gastrointestinal tract when bioavailability is maximized, and pre-systemic degradation is minimized. In some embodiments, at least one additional agent can be included to facilitate absorption of the amphiphilic conjugate. In certain embodiments, diluents, flavorings, low melting point waxes, vegetable oils, lubricants, suspending agents, tablet disintegrating agents, and binders can also be employed.

In some embodiments, a pharmaceutical composition can involve an effective quantity of an amphiphilic conjugate in a mixture with non-toxic excipients which are suitable for the manufacture of tablets. In some embodiments, by dissolving the tablets in sterile water, or another appropriate vehicle, solutions can be prepared in unit-dose form. In some embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled m the art, including formulations involving an amphiphilic conjugate in sustained- or controlled-delivery formulations. In some embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In some embodiments, sustained-release preparations can include semipermeable polymer matrices in the form of shaped articles, e.g., films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D (−)-3-hydroxybutyric acid (EP 133,988). In some embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In some embodiments, this can be accomplished by filtration through sterile filtration membranes. In certain embodiments, where the composition is lyophilized, sterilization using this method can be conducted either prior to or following lyophilization and reconstitution. In some embodiments, the composition for parenteral administration can be stored in lyophilized form or in a solution. In some embodiments, parenteral compositions generally are placed into a container having a sterile access port, for example, an intravenous solution bag or vial having a stopper pierceable by a hypodermic injection needle.

In some embodiments, once the pharmaceutical composition has been formulated, it can be stored in sterile vials as a solution, suspension, gel, emulsion, solid, or as a dehydrated or lyophilized powder. In some embodiments, such formulations can be stored either in a ready-to-use form or in a form (e.g., lyophilized) that is reconstituted prior to administration.

In some embodiments, kits are provided for producing a single-dose administration unit. In some embodiments, the kit can contain both a first container having a dried protein and a second container having an aqueous formulation. In some embodiments, kits containing single and multi-chambered pre-filled syringes (e.g., liquid syringes and syringes containing a lyophilized therapeutic) are included.

In some embodiments, the effective amount of a pharmaceutical composition including an amphiphilic conjugate to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an amphiphilic conjugate is being used, the route of administration, and the size (body weight, body surface or organ size) and/or condition (the age and general health) of the patient. In some embodiments, the clinician can titer the dosage and modify the route of administration to obtain the optimal therapeutic effect.

In some embodiments, the frequency of dosing will take into account the pharmacokinetic parameters of the amphiphilic conjugate, in the formulation used. In some embodiments, a clinician will administer the composition until a dosage is reached that achieves the desired effect. In some embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In some embodiments, appropriate dosages can be ascertained through use of appropriate dose-response data.

In some embodiments, the route of administration of the pharmaceutical composition is in accord with known methods, e.g., orally, through injection by intravenous, intraperitoneal, intracerebral (intra-parenchymal), intracerebroventricular, intramuscular, subcutaneously, intra-ocular, intraarterial, intraportal, or intralesional routes, by sustained release systems or by implantation devices. In certain embodiments, the compositions can be administered by bolus injection or continuously by infusion, or by implantation device. In certain embodiments, individual elements of the combination therapy may be administered by different routes.

In some embodiments, the composition can be administered locally via implantation of a membrane, sponge, or another appropriate material onto which the desired molecule has been absorbed or encapsulated. In some embodiments, where an implantation device is used, the device can be implanted into any suitable tissue or organ, and delivery of the desired molecule can be via diffusion, timed-release bolus, or continuous administration. In some embodiments, it can be desirable to use a pharmaceutical composition including an amphiphilic conjugate in an ex vivo manner. In such instances, cells, tissues and/or organs that have been removed from the patient are exposed to a pharmaceutical composition including an amphiphilic conjugate, after which the cells, tissues and/or organs are subsequently implanted back into the patient.

In some embodiments, an amphiphilic conjugate can be delivered by implanting certain cells that have been genetically engineered, using methods such as those described herein, to express and secrete the polypeptides. In some embodiments, such cells can be animal or human cells, and can be autologous, heterologous, or xenogeneic. In some embodiments, the cells can be immortalized. In some embodiments, in order to decrease the chance of an immunological response, the cells can be encapsulated to avoid infiltration of surrounding tissues. In some embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Methods

In some embodiments, the disclosure provides methods of expanding an immune cell (e.g., a T cell) expressing an EGFR receptor (e.g., an EGFR receptor encoded by a sequence of SEQ ID NO: 7, 8, 9, or 10) in vivo in a subject, including administering a composition including an amphiphilic lipid conjugate described herein.

In some embodiments, the disclosure provides methods of stimulation proliferation of an immune cell (e.g., a T cell) expressing and EGFR receptor (e.g., an EGFR receptor encoded by a sequence of SEQ ID NO: 7, 8, 9, or 10) in vivo in a subject, including administering a composition having an amphiphilic lipid conjugate described herein.

In some embodiments, the disclosure provides methods for treating a subject having a disease, disorder or condition associated with expression or elevated expression of an antigen, including administering to the subject an immune cell (e.g., a T cell) expressing an EGFR receptor (e.g., an EGFR receptor encoded by a sequence of SEQ ID NO: 7, 8, 9, or 10), and an amphiphilic lipid conjugate.

In some embodiments, the subject is administered the immune cell (e.g., a T cell) expressing an EGFR receptor (e.g., an EGFR receptor encoded by a sequence of SEQ ID NO: 7, 8, 9, or 10) prior to receiving the amphiphilic lipid conjugate. In some embodiments, the subject is administered the immune cell (e.g., a T cell) expressing an EGFR receptor (e.g., an EGFR receptor encoded by a sequence of SEQ ID NO: 7, 8, 9, or 10) after receiving the amphiphilic lipid conjugate. In some embodiments, the subject is administered the immune cell (e.g., a T cell) expressing an EGFR (e.g., an EGFR receptor encoded by a sequence of SEQ ID NO: 7, 8, 9, or 10) receptor and the amphiphilic lipid conjugate sequentially or simultaneously.

In some embodiments, the disclosure provides a method of stimulating an immune response to a target cell population or target tissue in a subject including administering to the subject an amphiphilic ligand conjugate including a lipid and a peptide having the amino acid sequence of SEQ ID NO: 1 or 2 and, optionally, a linker. In some embodiments, the method of stimulating an immune response to a target cell population or target tissue in a subject including administering to the subject an amphiphilic ligand conjugate including a lipid and a peptide having the amino acid sequence of SEQ ID NO: 1 or 2 and, optionally, a linker, does not include administering an immune cell (e.g., a T cell) expressing an EGFR receptor (e.g., an EGFR receptor encoded by a sequence of SEQ ID NO: 7, 8, 9, or 10) to the subject. Conjugating a cargo, such as an EGFR peptide including an amino acid sequence of SEQ ID NO: 5, to an albumin-binding domain can increase delivery and accumulation of the cargo to the lymph nodes, as described in U.S. Pat. No. 9,107,904 which is incorporated herein by reference in its entirety.

Methods for measuring expansion or proliferation of cells are known in the art. For example, the number of cells can be measured by introducing a dye (e.g., crystal violet) into cells, and measuring the dilution of the dye over time. Dilution indicates cell proliferation.

### Cancer and Cancer Immunotherapy

In some embodiments, the amphiphilic ligand conjugate and modified immune cells (e.g., EGFR modified T cells) described herein, are useful for treating a disorder associated with abnormal apoptosis or a differentiative process (e.g., cellular proliferative disorders (e.g., hyperproliferative disorders) or cellular differentiative disorders, such as cancer). Non-limiting examples of cancers that are amenable to treatment with the methods of the present invention are described below.

Examples of cellular proliferative and/or differentiative disorders include cancer (e.g., carcinoma, sarcoma, metastatic disorders, or hematopoietic neoplastic disorders, e.g., leukemias). A metastatic tumor can arise from a multitude of primary tumor types, including but not limited to those of prostate, colon, lung, breast, bladder, rectum, stomach, skin, kidney, cervix, and liver. Accordingly, the compositions used herein including an amphiphilic ligand conjugate can be administered to a patient who has cancer.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasm” are used to refer to malignancies of the various organ systems, including those affecting the lung, breast, brain, stomach, liver, skin, thyroid, lymph glands and lymphoid tissue, gastrointestinal organs, and the genitourinary tract (e.g., bladder, kidney, and cervix), as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The amphiphilic ligand conjugate can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon, and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid, or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

It will be appreciated by those skilled in the art that amounts for an amphiphilic conjugate and immune cells (e.g., a T cells) modified with and EGFR receptor (e.g., an EGFR receptor encoded by a sequence of SEQ ID NO: 7, 8, 9, or 10) that are sufficient to reduce tumor growth and size, or a therapeutically effective amount, will vary not only on the particular compound or composition selected, but also with the route of administration, the nature of the condition being treated, and the age and condition of the patient, and will ultimately be at the discretion of the patient's physician or pharmacist. The length of time during which the compound used in the instant method will be given varies on an individual basis.

In some embodiments, the disclosure provides methods of reducing or decreasing the size of a tumor or inhibiting a tumor growth in a subject in need thereof, including administering to the subject an amphiphilic lipid conjugate and a modified immune cell (e.g., a T cell) described herein to a subject. In some embodiments, the disclosure provides methods for inducing an anti-tumor response in a subject with cancer, including administering to the subject an amphiphilic lipid conjugate and an immune cell (e.g., a T cell) modified with an EGFR receptor (e.g., an EGFR receptor encoded by a sequence of SEQ ID NO: 7, 8, 9, or 10) described herein to a subject.

In some embodiments, the disclosure provides methods for stimulating an immune response to a target cell population or target tissue expressing an antigen in a subject, including administering an immune cell (e.g., a T cell) expressing an EGFR receptor (e.g., an EGFR receptor encoded by a sequence of SEQ ID NO: 7, 8, 9, or 10) targeted to the peptide, and an amphiphilic lipid conjugate. In some embodiments, the immune response is a T cell, a TIL (e.g., T cell, B cell, or an NK cell), an NK cell, an NKT cell, a gdT cell, a macrophage, a neutrophil, a dendritic cell, a mast cell, an eosinophil, or a basophil mediated immune response. In some embodiments, the immune response is an anti-tumor immune response. In some embodiments, the target cell population or target tissue is tumor cells or tumor tissue.

It will be appreciated by those skilled in the art that reference herein to treatment extends to prophylaxis as well as the treatment of the noted cancers and symptoms.

### Kits

A kit can include an amphiphilic ligand conjugate, including a peptide having the amino acid sequence of SEQ ID NO: 1 or 2, and an immune cell (e.g., a T cell) modified with an EGFR receptor (e.g., one encoded by the sequence of SEQ ID NO: 7, 8, 9, or 10), as disclosed herein, and instructions for use. The kits may include, in a suitable container, the amphiphilic ligand conjugate, immune cell (e.g., a T cell) modified with an EGFR receptor (e.g., an EGFR receptor encoded by a sequence of SEQ ID NO: 7, 8, 9, or 10), one or more controls, and various buffers, reagents, enzymes and other standard ingredients well known in the art. In some embodiments, the kits further include an adjuvant. Accordingly, in some embodiments, the amphiphilic ligand conjugate and the immune cell (e.g., a T cell) modified with an EGFR receptor (e.g., an EGFR receptor encoded by a sequence of SEQ ID NO: 7, 8, 9, or 10) are in the same vial. In some embodiments, the amphiphilic ligand conjugate and the immune cell (e.g., a T cell) modified with an EGFR receptor (e.g., an EGFR receptor encoded by a sequence of SEQ ID NO: 7, 8, 9, or 10) are in separate vials. Furthermore, in some embodiments, the amphiphilic ligand conjugate and adjuvant are in the same vial. In some embodiments, the amphiphilic ligand conjugate and adjuvant are in separate vials. In some embodiments, the immune cell (e.g., a T cell) modified with an EGFR receptor (e.g., an EGFR receptor encoded by a sequence of SEQ ID NO: 7, 8, 9, or 10) and adjuvant are in the same vial. In some embodiments, the immune cell (e.g., a T cell) modified with an EGFR receptor (e.g., an EGFR receptor encoded by a sequence of SEQ ID NO: 7, 8, 9, or 10) and the adjuvant are in separate vials.

The container can include at least one vial, well, test tube, flask, bottle, syringe, or other container means, into which an amphiphilic ligand conjugate may be placed, and in some instances, suitably aliquoted. When an additional component is provided, the kit can contain additional containers into which this compound may be placed. The kits can also include a means for containing an amphiphilic ligand conjugate, an immune cell (e.g., a T cell) modified with an EGFR receptor (e.g., an EGFR receptor encoded by a sequence of SEQ ID NO: 7, 8, 9, or 10), and any other reagent containers in close confinement for commercial sale. Such containers may include injection or blow-molded plastic containers into which the desired vials are retained. Containers and/or kits can include labeling with instructions for use and/or warnings.

In some embodiments, the disclosure provides a kit including a container including a composition including an amphiphilic ligand conjugate, an immune cell (e.g., a T cell) modified with an EGFR receptor (e.g., an EGFR receptor encoded by a sequence of SEQ ID NO: 7, 8, 9, or 10), an optional pharmaceutically acceptable carrier, and a package insert including instructions for administration of the composition for treating or delaying progression of cancer in an individual receiving therapy with an immune cell (e.g., a T cell) expressing an EGFR receptor (e.g., an EGFR receptor encoded by a sequence of SEQ ID NO: 7, 8, 9, or 10), wherein the amphiphilic ligand conjugate includes a lipid, a peptide having the amino acid sequence of SEQ ID NO: 1 or 2, and optionally a linker. In some embodiments, the kit further includes an adjuvant and instructions for administration of the adjuvant for treating or delaying progression of cancer in an individual receiving therapy with an immune cell (e.g., a T cell) modified with an EGFR receptor (e.g., an EGFR receptor encoded by a sequence of SEQ ID NO: 7, 8, 9, or 10).

In some embodiments, the disclosure provides a kit including a medicament including a composition including an amphiphilic ligand conjugate, an immune cell (e.g., a T cell) modified with an EGFR receptor (e.g., an EGFR receptor encoded by a sequence of SEQ ID NO: 7, 8, 9, or 10), an optional pharmaceutically acceptable carrier, and a package insert including instructions for administration of the medicament alone or in combination with a composition including an adjuvant and an optional pharmaceutically acceptable carrier, for treating or delaying progression of cancer, wherein the amphiphilic ligand conjugate includes a lipid, a peptide having the amino acid sequence of SEQ ID NO: 1 or 2, and optionally a linker.

In some embodiments, the disclosure provides a kit including a container including a composition including an amphiphilic ligand conjugate, an immune cell (e.g., a T cell) modified with an EGFR receptor (e.g., an EGFR receptor encoded by a sequence of SEQ ID NO: 7, 8, 9, or 10), an optional pharmaceutically acceptable carrier, and a package insert including instructions for administration of composition vaccine for expanding an immune cell (e.g., a T cell) expressing an EGFR receptor (e.g., an EGFR receptor encoded by a sequence of SEQ ID NO: 7, 8, 9, or 10) in a subject, wherein the amphiphilic ligand conjugate includes a lipid, a peptide having the amino acid sequence of SEQ ID NO: 1 or 2, and optionally a linker. In some embodiments, the kit further includes an adjuvant and instructions for administration of the adjuvant for expanding an immune cell (e.g., a T cell) modified with an EGFR receptor (e.g., an EGFR receptor encoded by a sequence of SEQ ID NO: 7, 8, 9, or 10).

### EXAMPLES

Below are examples of specific embodiments for making the constructs and carrying out the methods described herein. These examples are provided for illustrative purposes only and are not intended to limit the scope of the present invention in any way.

### Example 1. Construction of the EGFR Amphiphiles

EGFR peptides containing an internal disulfide bond were produced by solid phase synthesis (Almac). For peptides with an N-terminal amphiphile conjugate the peptides contained an ectopic Cys-thiozolidine group (SEQ ID NO: 2). For peptides with a C-terminal amphiphile conjugate the peptides contained an orthogonally protected Lys-thiozolidine group (SEQ ID NO: 1). Deprotecting these groups enabled conjugation with a 1, 2-Distearoyl-sn-glycero-3-phosphoethanolamine-PEG (DSPE-PEG)-maleimide group. N-methylsuccinimide (NMS) was used as a soluble control in place of an amphiphile (SEQ ID NOs: 3 and 4). For the NMS control, this DSPE-PEG-maleimide group contained only a methyl group. For the amphiphile conjugates, this DSPE-PEG-maleimide group contained a PEG24 linker and an amphiphile group. The compounds were synthesized as a trifluoroacetic acid salt with a purity of ≥95%. Amphiphile conjugated peptides and NMS controls were dissolved in sterile ultrapure water for further experiments.

### Example 2. Transduction of Human and Mouse Cells with an Amphiphile Conjugate

### B16F10 Transduction

### Production of EGFRviii-eGFP-fLUC Retrovirus

GP2-293 retroviral packaging cells were seeded at 80% confluence in a T175 flask and transfected with 15 μg of EGFRviii-eGFP-fLUC plasmid using Lipofectamine 3000 transfection reagent (Invitrogen). Transfection culture supernatants were collected at day 1 post transfection and at day 2 post transfection and stored at 4° C. until ultracentrifugation. Retrovirus particles were concentrated by ultracentrifugation at 25,000 rpm for 2 hours at 4° C. Supernatant was carefully decanted and retrovirus particles were resuspended in 400 μL DMEM media. Day 1 and day 2 concentrated samples were pooled for a final volume of 800 UL and stored at −80° C.

### Transduction of B16F10

Viable (≥95%) B16F10 cells were seeded at a density of 0.3×10⁶ cells per well of a retronectin-coated 6-well plate for 16-18 hr until confluent (approximately 1×10⁶ cells per well). Each well was transduced with 100 μL of concentrated EGFRviii-eGFP-fLUC retrovirus by spinoculation at 3,200 rpm for 90 min at 30° C. Plates were incubated at 37° C. for 24 hr and were transduced again with 100 μL of concentrated EGFRviii-eGFP-fLUC retrovirus by spinoculation at 3,200 rpm for 90 min at 30° C. Plates were incubated at 37° C. for 72 hr. Transduction efficiency was calculated using flow cytometry by gating on viable singlet cells and quantifying the percentage of eGFP-expressing cells in the FITC channel as compared to untransduced (UTD) B16F10 cells. fLUC expression was determined using the ONE-Glo luciferase assay system (Promega) and bioluminescence was quantified in a plate reader as compared to untransduced B16F10 cells. Transduced cells were maintained in cell culture and passaged for 2 weeks to confirm stable integration of the eGFP-fLUC construct. Limiting dilution was performed to select for clones of B16F10-EGFRviii-eGFP-fLUC that had higher % of GFP expression. B16F10 cells that were ≥95% GFP+ were used for subsequent experiments.

### Mouse T Cell Transduction

Phoenix-ECO retroviral packaging cells were seeded at 80%-90% confluence on a 100 mm dish (approximately 2×10⁶ cells). Cells were transfected with 4.3 μg of EGFR receptor or mCherry plasmid using Lipofectamine 3000 transfection reagent (Invitrogen). Transfection plates were cultured for 16-18 hr and cell culture media was replaced with fresh DMEM and plates were cultured for an additional 16-18 hr. At day 3 post-transfection, cells were examined under a fluorescence microscope to confirm mCherry expression. Supernatants were filtered through a 0.2 μm syringe filter and used at day 3 and day 4 to transduce mouse T cells.

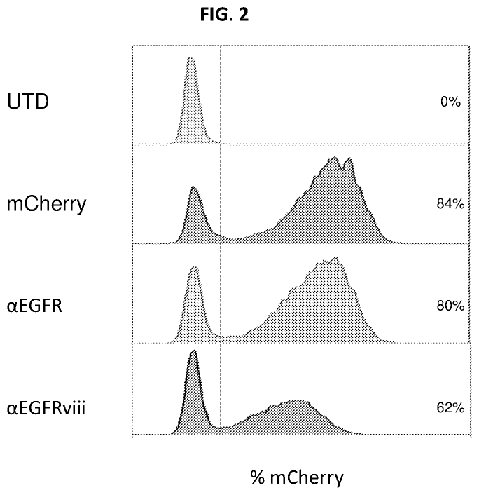

Mouse T cells were isolated from the spleens of C57-B6J mice using a EasySep mouse T cell isolation kit (Stemcell Technologies). Viable (>95%) isolated mouse T cells were activated in the presence of CD3/CD28 Dynabeads (Gibco) at a ratio of 2:1 beads: T cells and 250 IU of IL2. T cells were cultured at 37° C. for 24 hr at a density of 1×10⁶ cells/mL in a 6-well plate and received an additional 250 IU of IL2. At day 3 post-isolation T cells were resuspended in 1 mL at 3×10⁶ cells/well of a retronectin-coated 6 well plate together with 2 mL of filtered retrovirus supernatant. Mouse T cells were transduced by spinoculation at 3,200 rpm for 90 min at 30° C. and cultured for 24 hr at 37° C. Mouse T cells were transduced for a second time by spinoculation with filtered retrovirus supernatant at 3,200 rpm for 90 min at 30° C. and cultured for 72 hr at 37° C. Transduction efficiency was calculated using flow cytometry by gating on viable singlet cells and quantifying the percentage of mCherry-expressing cells in the PE-CF594 channel as compared to untransduced T cells (FIG. 2).

### U87-MG Transduction

Production of eGFP-fLUC Retrovirus

GP2-293 retroviral packaging cells were seeded at 80% confluence in a T175 flask and transfected with 15 μg of eGFP-fLUC plasmid using Lipofectamine 3000 transfection reagent (Invitrogen). Transfection culture supernatants were collected at day 1 post transfection and at day 2 post transfection and stored at 4° C. until ultracentrifugation. Retrovirus particles were concentrated by ultracentrifugation at 25,000 rpm for 2 hours at 4° C. Supernatant was carefully decanted and retrovirus particles were resuspended in 400 μL DMEM media. Day 1 and day 2 concentrated samples were pooled for a final volume of 800 μL and stored at −80° C.

### Transduction of U87-MG

Viable (≥95%) U87-MG cells were seeded at a density of 0.3×10⁶ cells per well of a retronectin-coated 6-well plate for 16-18 hr until confluent (approximately 1×10⁶ cells per well). Each well was transduced with 100 μL of concentrated eGFP-fLUC retrovirus by spinoculation at 3,200 rpm for 90 min at 30° C. Plates were incubated at 37° C. for 24 hr and were transduced again with 100 μL of concentrated eGFP-fLUC retrovirus by spinoculation at 3,200 rpm for 90 min at 30° C. Plates were incubated at 37° C. for 72 hr. Transduction efficiency was calculated using flow cytometry by gating on viable singlet cells and quantifying the percentage of eGFP-expressing cells in the FITC channel as compared to untransduced U87-MG cells. fLUC expression was determined using the ONE-Glo luciferase assay system (Promega) and bioluminescence was quantified in a plate reader as compared to untransduced U87-MG cells. Transduced cells were maintained in cell culture and passaged for 2 weeks to confirm stable integration of the eGFP-fLUC construct. U87-MG cells that were ≥95% GFP+ were used for subsequent experiments.

Production of EGFR Receptor or mCherry Retrovirus

Phoenix-AMPHO retroviral packaging cells were seeded at 80%-90% confluence on a 100 mm dish (approximately 2×10⁶ cells). Cells were transfected with 4.3 μg of EGFR receptor or mCherry plasmid using Lipofectamine 3000 transfection reagent (Invitrogen). Transfection plates were cultured for 16-18 hr and cell culture media was replaced with fresh DMEM and plates were cultured for an additional 16-18 hr. At day 3 post-transfection, cells were examined under a fluorescence microscope to confirm mCherry expression. Supernatants were filtered through a 0.2 μm syringe filter and used at day 3 and day 4 to transduce human T cells.

### Transduction of Human T Cells

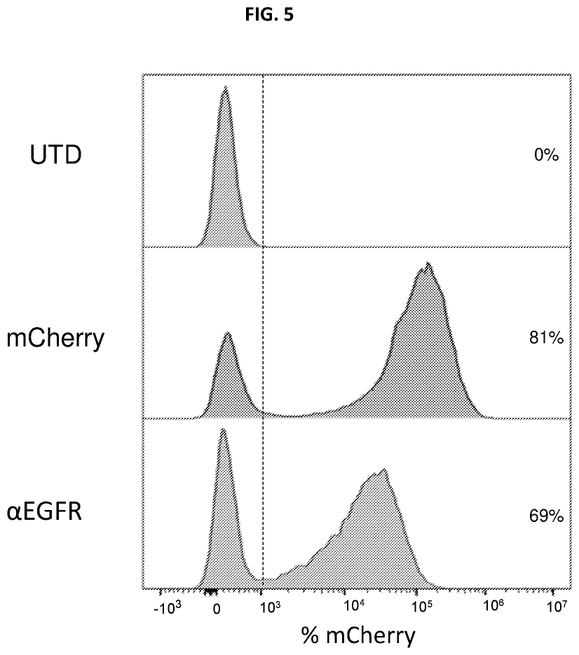

Human T cells were isolated from donor Leukopaks using an EasySep human T cell isolation kit (Stemcell Technologies). Viable (>95%) isolated human T cells were activated in the presence of CD3/CD28 Dynabeads (Gibco) at a ratio of 2:1 beads: T cells and 250 IU of IL2. T cells were cultured at 37° C. for 24 hr at a density of 1×10⁶ cells/mL in a 6-well plate and received an additional 250 IU of IL2. At day 3 post-isolation T cells were resuspended in 1 mL at 3×10⁶ cells/well of a retronectin-coated 6 well plate together with 2 mL of filtered retrovirus supernatant. Human T cells were transduced by spinoculation at 3,200 rpm for 90 min at 30° C. and cultured for 24 hr at 37° C. Human T cells were transduced for a second time by spinoculation with filtered retrovirus supernatant at 3,200 rpm for 90 min at 30° C. and cultured for 72 hr at 37° C. Transduction efficiency was calculated using flow cytometry by gating on viable singlet cells and quantifying the percentage of mCherry-expressing cells in the PE-CF594 channel as compared to untransduced T cells (FIG. 5).

### Example 3. Stimulation of T Cells In Vitro

### Mouse Cell Killing Assay

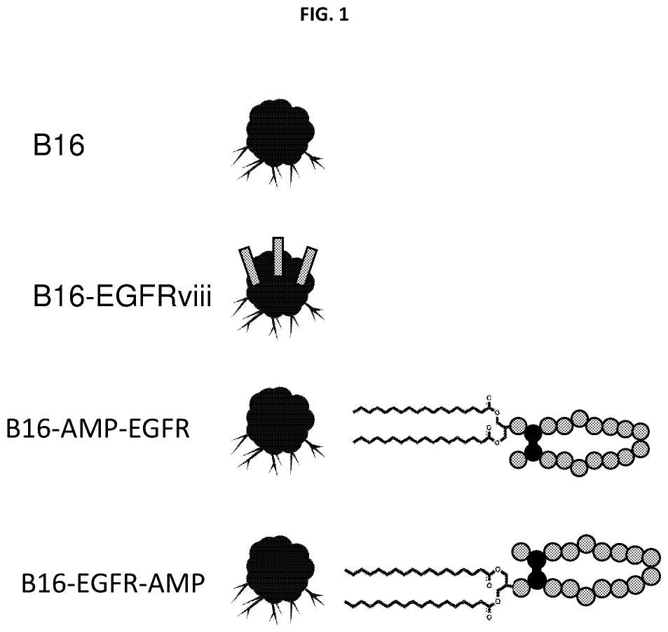

B16F10 cells (>95% viability, ≥95% GFP expression) and B16F10 cells expressing EGFRviii (>95% viability, ≥95% GFP) were seeded at 5×10⁴ cells/well of a 96 well plate and cultured for 3 hr at 37° C. Cells were then labeled with either 400 nmol of the EGFR amphiphilic conjugate for 2 hr at 37° C. or media (no amphiphile) (FIG. 1). The wells were next washed 2 times with media to flush out any excess or unbound EGFR amphiphilic conjugate. Mouse T-cells were resuspended at 1×10⁶ viable transduced cells/mL and the CD3/CD28 beads were removed using a magnet. De-beaded T cells were rested for 5 hr at 37° C. to return to a baseline activated state following magnetic bead activation.

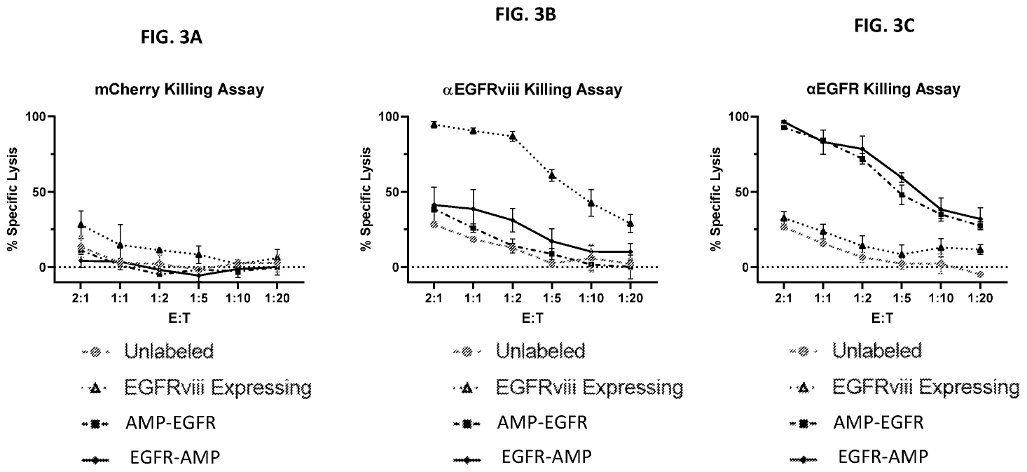

Unlabeled and amphiphile conjugate-labeled B16F10 cells, labeled with EGFRviii or amphiphilic EGFR peptide with the amphiphile located at the N-terminal or C-terminal end of the peptide, were co-cultured with rested mouse T cells at the desired effector: target ratios (2:1, 1:1, 1:2, 1:5, 1:10, 1:20) in triplicate. Killing assay plates were incubated for 16-18 hr at 37° C. Following co-culture, the plates were spun down at 1,200 rpm for 5 min at 25° C. and the supernatant was decanted. Luciferase expression was determined using the ONE-Glo luciferase assay system (Promega) and bioluminescence was quantified in a plate reader. Killing was calculated as a relative % of living cells in co-cultures compared to fully viable target-only B16F10 cells and subtracted from 100 to get % of killed cells. The mean of 3 technical replicates of % killing was graphed for each effector: target ratio using Prism 8 (GraphPad) (FIGS. 3A-3C).

### Human Cell Killing Assay:

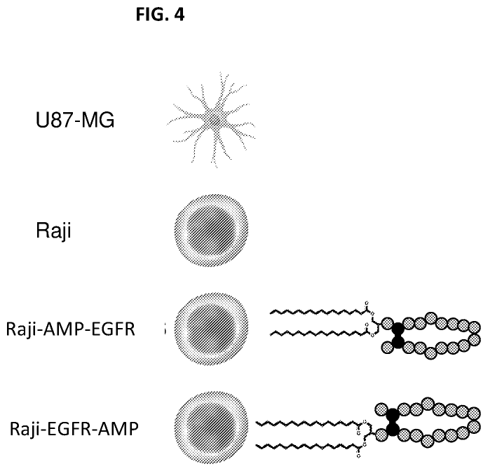

U87-MG cells (>95% viability, ≥95% GFP expression) and Raji cells (≥95% viability, ≥95% GFP) were seeded at 5×10⁴ cells/well of a 96 well plate and cultured for 3 hr at 37° C. Cells were then labeled with either 400 nmol of the EGFR amphiphilic conjugate for 2 hr at 37° C. or media (no amphiphile) (FIG. 4). The wells were next washed 2 times with media to flush out any excess or unbound EGFR amphiphilic conjugate. Human T cells were resuspended at 1×10⁶ viable transduced cells/mL and the CD3/CD28 beads were removed using a magnet. De-beaded T cells were rested for 5 hr at 37° C. to return to a baseline activated state following magnetic bead activation.

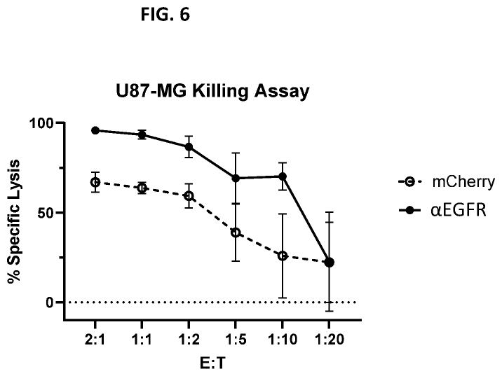

Unlabeled and amphiphile conjugate-labeled Raji cells and U87-MG cells were co-cultured with rested human T cells at the desired effector: target ratios (2:1, 1:1, 1:2, 1:5, 1:10, 1:20) in triplicate. Killing assay plates were incubated for 16-18 hr at 37° C. Following co-culture, the plates were spun down at 1,200 rpm for 5 min at 25° C. and the supernatant was decanted. Luciferase expression was determined using the ONE-Glo luciferase assay system (Promega) and bioluminescence was quantified in a plate reader. Killing was calculated as a relative % of living cells in co-cultures compared to fully viable target-only U87-MG and Raji cells and subtracted from 100 to get % of killed cells. The mean of 3 technical replicates of % killing was graphed for each effector: target ratio using Prism 8 (GraphPad) (FIG. 6).

Additionally, U87-MG cells (>95% viability, >95% GFP expression) and Raji cells (>95% viability, ≥95% GFP) were seeded at a density of 1×10⁶ cells per well of a 6-well plate for 3 hr. Raji cells were then labeled with either 400 nmol of the EGFR amphiphilic conjugate for 2 hr at 37° C. or media (no amphiphile). The wells were next washed 2 times with media to flush out any excess or unbound EGFR amphiphilic conjugate. Human T cells were resuspended at 1×10⁶ viable transduced cells/mL and the CD3/CD28 beads were removed using a magnet. De-beaded T cells were rested for 5 hr at 37° C. to return to a baseline activated state following magnetic bead activation. Target cells and human T cells were co-cultured at an effector: target ratio of 2:1 (2×10⁶ T cells: 1×10⁶ target cells) in a 6 well plate for 16-18 hr at 37° C. The plates were briefly spun at 1,200 rpm for 5 min at 25° C. and supernatants were carefully collected to run Luminex.

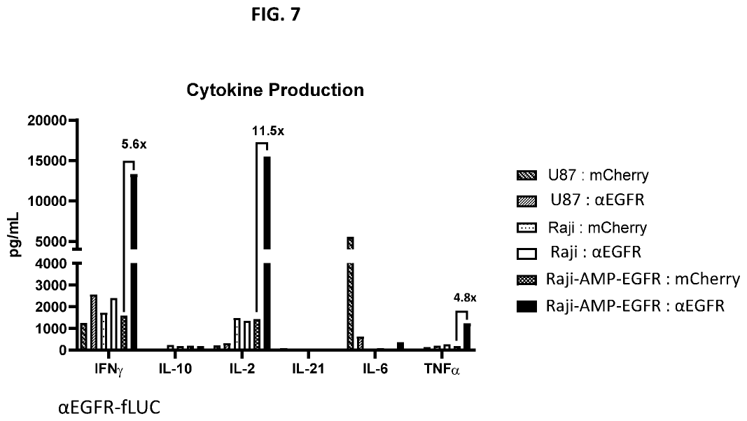

Cyokine levels were quantified using a Milliplex MAP human Th17 magnetic bead panel (Millipore Sigma) following the manufacturer's protocol. Magnetic bead mixes and standards and controls were all prepared following the manufacturer's instructions. 25 L of supernatants collected from target: T cell cultures were used for the experiment. The plate was incubated for 16-18 hr at 4° C. The plate was attached to a magnet and the supernatants were decanted and washed 2 times with the provided wash buffer. Wells were incubated with 25 μL of detection antibodies for 1 hr at 25° C. and followed by 25 μL of streptavidin-phycoerythrin for 30 min at 25° C. The plate was attached to a magnet and the supernatants were decanted and washed 2 times with the provided wash buffer and samples were read on a Luminex LX200 instrument using xPONENT software (FIG. 7).

### NUMBERED EMBODIMENTS

Some embodiments of the technology described herein can be defined according to any one of the following numbered embodiments.
1. An amphiphilic ligand conjugate, or a pharmaceutically acceptable salt thereof, comprising a lipid, an EGFR peptide, and, optionally, a linker.
2. The amphiphilic ligand conjugate, or pharmaceutically acceptable salt thereof, of embodiment 1, wherein the EGFR peptide consists of 7 to 40 amino acids.
3. The amphiphilic ligand conjugate, or pharmaceutically acceptable salt thereof, of embodiment 1 or 2, wherein the EGFR peptide is a binding ligand for an EGFR-specific antibody.
4. The amphiphilic ligand conjugate, or pharmaceutically acceptable salt thereof, of embodiment 3, wherein the EGFR-specific antibody is antibody 806.
5. The amphiphilic ligand conjugate, or pharmaceutically acceptable salt thereof, of any one of embodiments 1-4, wherein the EGFR peptide comprises an amino acid sequence of SEQ ID NO: 1, 2, or 5.
6. The amphiphilic ligand conjugate, or pharmaceutically acceptable salt thereof, of any one of embodiments 1-5, wherein the EGFR peptide is a cyclic peptide.
7. The amphiphilic ligand conjugate, or pharmaceutically acceptable salt thereof, of embodiment 5 or 6, wherein the peptide comprises an amino acid sequence identical to SEQ ID NO: 1.
8. The amphiphilic ligand conjugate, or pharmaceutically acceptable salt thereof, of embodiment 5 or 6, wherein the peptide comprises an amino acid sequence identical to SEQ ID NO: 2.
9. The amphiphilic ligand conjugate, or pharmaceutically acceptable salt thereof, of embodiment 5 or 6, wherein the peptide comprises an amino acid sequence identical to SEQ ID NO: 5.
10. The amphiphilic ligand conjugate, or pharmaceutically acceptable salt thereof, of any one of embodiments 1-9, wherein the lipid of the amphiphilic ligand conjugate is a diacyl lipid.
11. The amphiphilic ligand conjugate, or a pharmaceutically acceptable salt thereof, of embodiment 10, wherein the diacyl lipid of the amphiphilic ligand conjugate comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.
12. The amphiphilic ligand conjugate, or pharmaceutically acceptable salt thereof of embodiment 11, wherein the lipid is 1,2-distearoyl-sn-glycero-3-phosphoethanolamine (DSPE).
13. The amphiphilic ligand conjugate, or pharmaceutically acceptable salt thereof, of any one of embodiments 1-12, wherein the linker is present and is selected from the group consisting of a hydrophilic polymer, a string of hydrophilic amino acids, a polysaccharide, and an oligonucleotide, or a combination thereof.
14. The amphiphilic ligand conjugate, or pharmaceutically acceptable salt thereof, of embodiment 13, wherein the linker comprises “N” polyethylene glycol units, wherein N is between 24-50.
15. The amphiphilic ligand conjugate, or pharmaceutically acceptable salt thereof, of embodiment 14, wherein the linker comprises PEG24-amido-PEG24.
16. The amphiphilic ligand conjugate, or pharmaceutically acceptable salt thereof, of any one of embodiments 1-15, wherein the amphiphilic ligand conjugate is trafficked to a lymph node.
17. The amphiphilic ligand conjugate, or pharmaceutically acceptable salt thereof, of embodiment 16, wherein the amphiphilic ligand conjugate is trafficked to an inguinal lymph node or an axillary lymph node.
18. The amphiphilic ligand conjugate, or pharmaceutically acceptable salt thereof, of embodiment 16 or 17, wherein the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.
19. The amphiphilic ligand conjugate, or pharmaceutically acceptable salt thereof, of any one of embodiments 1-15, wherein the lipid is conjugated directly or via a linker to the N-terminus of the EGFR peptide.
20. The amphiphilic ligand conjugate, or pharmaceutically acceptable salt thereof, of any one of embodiments 1-15, wherein the lipid is conjugated directly or via a linker to the C-terminus of the EGFR peptide.
21. A method of stimulating an immune response to a target cell population or target tissue in a subject, the method comprising administering to the subject (1) an amphiphilic ligand conjugate, comprising a lipid, an EGFR peptide, and, optionally, a linker, and (2) an immune cell modified with an EGFR receptor, wherein the EGFR receptor binds the EGFR peptide of the amphiphilic ligand conjugate.
22. The method of embodiment 21, wherein the EGFR peptide comprises an amino acid sequence of SEQ ID NO: 1, 2, or 5.
23. The method of embodiment 21 or 22, further comprising administering an adjuvant to the subject.
24. The method of embodiment 23, wherein the adjuvant is an amphiphilic oligonucleotide conjugate comprising an immunostimulatory oligonucleotide conjugated to a lipid, with or without a linker.
25. The method of any one of embodiments 21-24, wherein the EGFR peptide is a cyclic peptide.
26. The method of any one of embodiments 21-25, wherein the peptide comprises an amino acid sequence identical to SEQ ID NO: 1.
27. The method of any one of embodiments 21-25, wherein the peptide comprises an amino acid sequence identical to SEQ ID NO: 2.
28. The method of any one of embodiments 21-25, wherein the peptide comprises an amino acid sequence identical to SEQ ID NO:5.
29. The method of any one of embodiments 21-28, wherein the EGFR receptor is an EGFRviii receptor.
30. The method of any one of embodiments 21-29, wherein the lipid of the amphiphilic ligand conjugate inserts into a cell membrane under physiological conditions, binds albumin under physiological conditions, or both.
31. The method of any one of embodiments 21-30, wherein the lipid of the amphiphilic ligand conjugate is a diacyl lipid.
32. The method of embodiment 31, wherein the diacyl lipid of the amphiphilic ligand conjugate comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units. 33. The method of embodiment 32, wherein the lipid is 1,2-distearoyl-sn-glycero-3-phosphoethanolamine (DSPE).
34. The method of any one of embodiments 21-33, wherein the linker is selected from the group consisting of a hydrophilic polymer, a string of hydrophilic amino acids, a polysaccharide, and an oligonucleotide, or a combination thereof.
35. The method of embodiment 34, wherein the linker comprises “N” polyethylene glycol units, wherein N is between 24-50.
36. The method of embodiments 35, wherein the linker comprises PEG24-amido-PEG24.
37. The method of any one of embodiments 21-36, wherein the amphiphilic ligand conjugate is trafficked to a lymph node. 38. The method of embodiment 37, wherein the amphiphilic ligand conjugate is trafficked to an inguinal lymph node or an axillary lymph node. 39. The method of embodiment 37 or 38, wherein the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.
40. The method of any one of embodiments 21-39, wherein the immune cell is a T cell, a B cell, a natural killer (NK) cell, a macrophage, a neutrophil, a dendritic cell, a mast cell, an eosinophil, or a basophil.
41. The method of embodiment 40, wherein the immune cell is a T cell.
42. The method of any one of embodiments 21-41, wherein the immune response is an anti-tumor immune response.
43. The method of any one of embodiments 21-42, wherein the target cell population or the target tissue is a tumor cell population or a tumor tissue.
44. The method of any one of embodiments 21-43, wherein the method comprises reducing or decreasing the size of the tumor tissue or inhibiting growth of the tumor cell population or the tumor tissue in the subject.
45. The method of any one of embodiments 21-44, wherein the method comprises activating the immune cell, expanding the immune cell, and/or increasing proliferation of the immune cell.
46. The method of any one of embodiments 21-45, wherein the subject has a disease, a disorder, or a condition associated with expression or elevated expression of the antigen.
47. The method of any one of embodiments 21-46, wherein the subject is lymphodepleted prior to the administration of the amphiphilic lipid conjugate.
48. The method of embodiment 47, wherein the lymphodepletion is by sublethal irradiation.
49. The method of any one of embodiments 21-48, wherein the subject is administered the amphiphilic ligand conjugate prior to receiving the immune cell modified with an EGFR receptor.
50. The method of any one of embodiments 21-49, wherein the subject is administered the amphiphilic ligand conjugate after receiving the immune cell modified with an EGFR receptor.
51. The method of any one of embodiments 21-50, wherein amphiphilic ligand conjugate of and the receiving immune cell modified with an EGFR receptor are administered simultaneously.
52. The method of any one of embodiments 21-51, wherein the lipid is conjugated directly or via a linker to the N-terminus of the EGFR peptide.
53. The method of any one of embodiments 21-51, wherein the lipid is conjugated directly or via a linker to the C-terminus of the EGFR peptide.
54. A kit comprising (1) an amphiphilic ligand conjugate or pharmaceutically acceptable salt thereof of any one of embodiments 1-20, and (2) an immune cell modified with an EGFR receptor, wherein the EGFR receptor binds the EGFR peptide of the amphiphilic ligand conjugate.
55. The kit of embodiment 54, wherein the EGFR receptor is an EGFRviii receptor.

Listing of Sequences

SEQ ID NO: 1
ACGADSYEMEEDGVRKCK(AMP-Cys), Internal disulfide bridge (Cys287-Cys302)
SEQ ID NO: 2
(AMP-Cys)ACGADSYEMEEDGVRKCK, Internal disulfide bridge (Cys287-Cys302)
SEQ ID NO: 3
ACGADSYEMEEDGVRKCK(NMS-Cys), Internal disulfide bridge (Cys287-Cys302)
SEQ ID NO: 4
(NMS-Cys)ACGADSYEMEEDGVRKCK, Internal disulfide bridge (Cys287-Cys302)
SEQ ID NO: 5
ACGADSYEMEEDGVRKCK
SEQ ID NO: 6
5′-L-GnTCCATGACGTTCCTGACGTT-3′ (wherein “L” is a lipophilic compound, such as diacyl lipid, “Gn” is
a guanine repeat linker and “n” represents 1, 2, 3, 4, or 5)
SEQ ID NO: 7-Human a806 CAR
pELI0089 MSGV-Kozak hCD8 leader 806 VHVL CD8 hinge and TMD 41BB CD3z mCHERRY WPRE
aatgaaagaccccacctgtaggtttggcaagctagcttaagtaacgccattttgcaaggcatggaaaatacataactgagaatagagaagttcaga
tcaaggttaggaacagagagacagcagaatatgggccaaacaggatatctgtggtaagcagttcctgccccggctcagggccaagaacagatg
gtccccagatgcggtcccgccctcagcagtttctagagaaccatcagatgtttccagggtgccccaaggacctgaaatgaccctgtgccttatttgaa
ctaaccaatcagttcgcttctcgcttctgttcgcgcgcttctgctccccgagctcaataaaagagcccacaacccctcactcggcgcgccagtcctccg
atagactgcgtcgcccgggtacccgtattcccaataaagcctcttgctgtttgcatccgaatcgtggactcgctgatccttgggagggtctcctcagatt
gattgactgcccacctcgggggtctttcatttggaggttccaccgagatttggagacccctgcccagggaccaccgacccccccgccgggaggtaag
ctggccagcggtcgtttcgtgtctgtctctgtctttgtgcgtgtttgtgccggcatctaatgtttgcgcctgcgtctgtactagttagctaactagctc
tgtatctggcggacccgtggtggaactgacgagttcggaacacccggccgcaaccctgggagacgtcccagggacttcgggggccgtttttgtggcccg
acctgagtcctaaaatcccgatcgtttaggactctttggtgcaccccccttagaggagggatatgtggttctggtaggagacgagaacctaaaacagtt
cccgcctccgtctgaatttttgctttcggtttgggaccgaagccgcgccgcgcgtcttgtctgctgcagcatcgttctgtgttgtctctgtctgactgt
gtttctgtatttgtctgaaaatatgggcccgggctagcctgttaccactcccttaagtttgaccttaggtcactggaaagatgtcgagcggatcgctca
caaccagtcggtagatgtcaagaagagacgttgggttaccttctgctctgcagaatggccaacctttaacgtcggatggccgcgagacggcacctttaa
ccgagacctcatcacccaggttaagatcaaggtcttttcacctggcccgcatggacacccagaccaggtcccctacatcgtgacctgggaagccttggc
ttttgacccccctccctgggtcaagccctttgtacaccctaagcctccgcctcctcttcctccatccgccccgtctctcccccttgaacctcctcgttc
gaccccgcctcgatcctccctttatccagccctcactccttctctaggcgcccccatatggccatatgagatcttatatggggcacccccgccccttgt
aaacttccctgaccctgacatgacaagagttactaacagcccctctctccaagctcacttacaggctctctacttagtccagcacgaagtctggagac
ctctggcggcagcctaccaagaacaactggaccgaccggtggtacctcacccttaccgagtcggcgacacagtgtgggtccgccgacaccagactaaga
acctagaacctcgctggaaaggaccttacacagtcctgctgaccacccccaccgccctcaaagtagacggcatcgcagcttggatacacgccgcccacg
tgaaggctgccgaccccgggggtggaccatcctctagccctcgagaagcttgccaccATGGCCCTGCCTGTGACAGCCCTGCT
GCTGCCTCTGGCTCTGCTGCTGCATGCCGCTAGACCCGATGTCCAGCTGCAAGAGTCTGGCCCTAG
CCTGGTCAAGCCTAGCCAGAGCCTGAGCCTGACATGTACCGTGACCGGCTACAGCATCACCAGCGA
CTTCGCCTGGAACTGGATCAGACAGTTCCCCGGCAACAAGCTGGAATGGATGGGCTACATCAGCTA
CAGCGGCAACACCCGGTACAACCCCAGCCTGAAGTCCCGGATCTCCATCACCAGAGACACCAGCAA
GAACCAGTTCTTCCTGCAGCTGAACAGCGTGACCATCGAGGACACCGCCACCTACTACTGTGTGAC
AGCCGGCAGAGGCTTCCCTTATTGGGGACAGGGAACCCTGGTCACAGTGTCTGCTGGTGGCGGAG
GATCTGGCGGAGGCGGATCTTCTGGCGGTGGCTCTGATATCCTGATGACACAGAGCCCCAGCAGCA
TGTCTGTGTCCCTGGGCGATACCGTGTCCATCACCTGTCACAGCAGCCAGGACATCAACAGCAACA
TCGGCTGGCTGCAGCAGAGGCCTGGCAAGTCTTTTAAGGGCCTGATCTACCACGGCACCAACCTGG
ATGATGAGGTGCCCAGCAGATTTTCCGGCTCTGGAAGCGGAGCCGACTACTCCCTGACAATCAGCA
GCCTGGAAAGCGAGGACTTCGCCGATTACTACTGCGTGCAGTACGCCCAGTTTCCTTGGACCTTTG
GAGGCGGCACAAAGCTGGAAATCAAGCGGACCACGACGCCAGCGCCGCGACCACCAACACCGGC
GCCCACCATCGCGTCGCAGCCCCTGTCCCTGCGCCCAGAGGCGTGCCGGCCAGCGGGGGGGGC
GCAGTGCACACGAGGGGGCTGGACTTCGCCTGTGATATCTACATCTGGGCGCCCTTGGCCGGGAC
TTGTGGGGTCCTTCTCCTGTCACTGGTTATCACCCTTTACTGCAAACGGGGCAGAAAGAAACTCCTG
TATATATTCAAACAACCATTTATGAGACCAGTACAAACTACTCAAGAGGAAGATGGCTGTAGCTGCCG
ATTTCCAGAAGAAGAAGAAGGAGGATGTGAACTGAGAGTGAAGTTCAGCAGGAGCGCAGACGCCCC
CGCGTACAAGCAGGGCCAGAACCAGCTCTATAACGAGCTCAATCTAGGACGAAGAGAGGAGTACGA
TGTTTTGGACAAGAGACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTACAGTGAGATTGGGATGA
AAGGCGAGCGCCGGAGGGGCAAGGGGCACGATGGCCTTTACCAGGGTCTCAGTACAGCCACCAAG
GACACCTACGACGCCCTTCACATGCAGGCCCTGCCCCCTCGCTCCGGAGGCGGCGGAGAGGGCAG
AGGAAGTCTTCTAACATGCGGTGACGTGGAGGAGAATCCCGGCCCTAGGATGGTCTCCAAAGGGGA
GGAAGACAACATGGCTATCATCAAAGAATTTATGAGATTTAAGGTCCATATGGAAGGCTCAGTGAAT
GGTCACGAATTCGAGATCGAAGGCGAAGGAGAGGGTCGGCCTTATGAGGGGACACAGACAGCTAA
ACTTAAGGTTACAAAAGGTGGACCATTGCCCTTTGCCTGGGACATCCTCAGTCCACAGTTTATGTAT
GGGAGCAAAGCCTATGTTAAGCATCCCGCAGACATCCCTGATTACCTTAAGCTTTCATTTCCCGAAG
GTTTCAAGTGGGAGAGGGTAATGAACTTTGAAGACGGGGGGGTTGTTACCGTTACACAGGATAGCA
GTCTTCAAGATGGCGAATTTATCTATAAGGTGAAACTGCGGGGCACCAACTTTCCCAGCGATGGGCC
TGTGATGCAAAAGAAAACAATGGGATGGGAAGCAAGTAGCGAAAGGATGTACCCAGAAGATGGTGC
CCTTAAGGGAGAGATCAAGCAACGGCTTAAACTGAAAGATGGTGGACACTACGATGCAGAAGTCAAA
ACAACTTACAAAGCCAAAAAGCCCGTTCAACTCCCAGGTGCCTACAACGTTAATATTAAGCTGGACAT
TACAAGTCATAATGAAGATTACACTATAGTTGAGCAGTACGAGCGGGCCGAGGGCAGGCACAGCAC
AGGTGGTATGGACGAACTGTATAAGTAACGCTCGACAATCAACCTCTGGATTACAAAATTTGTGAAA
GATTGACTGGTATTCTTAACTATGTTGCTCCTTTTACGCTATGTGGATACGCTGCTTTAATGCCTTTGT
ATCATGCTATTGCTTCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTTGCTGACGCAACCC
CCACTGGTTGGGGCATTGCCACCACCTGTCAGCTCCTTTCCGGGACTTTCGCTTTCCCCCTCCCTAT
TGCCACGGCGGAACTCATCGCCGCCTGCCTTGCCCGCTGCTGGACAGGGGCTCGGCTGTTGGGCA
CTGACAATTCCGTGGTGTTGTCGGGGAAGCTGACGTCCTTTCCATGGCTGCTCGCCTGTGTTGCCA
CCTGGATTCTGCGCGGGACGTCCTTCTGCTACGTCCCTTCGGCCCTCAATCCAGCGGACCTTCCTT
CCCGCGGCCTGCTGCCGGCTCTGCGGCCTCTTCCGCGTCTTCGCCTTCGCCCTCAGACGAGTCGG
ATCTCCCTTTGGGCCGCCTCCCCGCCTGgtcgacggtaccgcgggcccgggatccgataaaataaaagattttatttagtctcc
agaaaaaggggggaatgaaagaccccacctgtaggtttggcaagctagcttaagtaacgccattttgcaaggcatggaaaatacataactgaga
atagagaagttcagatcaaggttaggaacagagagacagcagaatatgggccaaacaggatatctgtggtaagcagttcctgccccggctcagg
gccaagaacagatggtccccagatgcggtcccgccctcagcagtttctagagaaccatcagatgtttccagggtgccccaaggacctgaaatgac
cctgtgccttatttgaactaaccaatcagttcgcttctcgcttctgttcgcgcgcttctgctccccgagctcaataaaagagcccacaacccctcactc
ggcgcgccagtcctccgatagactgcgtcgcccgggtacccgtgtatccaataaaccctcttgcagttgcatccgacttgtggtctcgctgttccttgg
gagggtctcctctgagtgattgactacccgtcagcgggggtctttcatgggtaacagtttcttgaagttggagaacaacattctgagggtaggagtcga
atattaagtaatcctgactcaattagccactgttttgaatccacatactccaatactcctgaaatccatcgatggagttcattatggacagcgcagaaa
gagctggggagaattgtgaaattgttatccgctcacaattccacacaacatacgagccggaagcataaagtgtaaagcctggggtgcctaatgagtgag
ctaactcacattaattgcgttgcgctcactgcccgctttccagtcgggaaacctgtcgtgccagctgcattaatgaatcggccaacgcgcggggagagg
cggtttgcgtattgggcgctcttccgcttcctcgctcactgactcgctgcgctcggtcgttcggctgcggcgagcggtatcagctcactcaaaggcggt
aatacggttatccacagaatcaggggataacgcaggaaagaacatgtgagcaaaaggccagcaaaaggccaggaaccgtaaaaaggccgcgt
tgctggcgtttttccataggctccgcccccctgacgagcatcacaaaaatcgacgctcaagtcagaggtggcgaaacccgacaggactataaaga
taccaggcgtttccccctggaagctccctcgtgcgctctcctgttccgaccctgccgcttaccggatacctgtccgcctttctcccttcgggaagcgtg
gcgctttctcatagctcacgctgtaggtatctcagttcggtgtaggtcgttcgctccaagctgggctgtgtgcacgaaccccccgttcagcccgaccgc
tgcgccttatccggtaactatcgtcttgagtccaacccggtaagacacgacttatcgccactggcagcagccactggtaacaggattagcagagcgagg
tatgtaggcggtgctacagagttcttgaagtggtggcctaactacggctacactagaagaacagtatttggtatctgcgctctgctgaagccagttacc
ttcggaaaaagagttggtagctcttgatccggcaaacaaaccaccgctggtagcggtggtttttttgtttgcaagcagcagattacgcgcagaaaaaa
aggatctcaagaagatcctttgatcttttctacggggtctgacgctcagtggaacgaaaactcacgttaagggattttggtcatgagattatcaaaaag
gatcttcacctagatccttttaaattaaaaatgaagttttaaatcaatctaaagtatatatgagtaaacttggtctgacagttaccaatgcttaatcag
tgaggcacctatctcagcgatctgtctatttcgttcatccatagttgcctgactccccgtcgtgtagataactacgatacgggagggcttaccatctgg
ccccagtgctgcaatgataccgcgagacccacgctcaccggctccagatttatcagcaataaaccagccagccggaagggccgagcgcagaagtggtcct
gcaactttatccgcctccatccagtctattaattgttgccgggaagctagagtaagtagttcgccagttaatagtttgcgcaacgttgttgccattgct
acaggcatcgtggtgtcacgctcgtcgtttggtatggcttcattcagctccggttcccaacgatcaaggcgagttacatgatcccccatgttgtgcaaa
aaagcggttagctccttcggtcctccgatcgttgtcagaagtaagttggccgcagtgttatcactcatggttatggcagcactgcataattctcttact
gtcatgccatccgtaagatgcttttctgtgactggtgagtactcaaccaagtcattctgagaatagtgtatgcggcgaccgagttgctcttgcccggcg
tcaatacgggataataccgcgccacatagcagaactttaaaagtgctcatcattggaaaacgttcttcggggcgaaaactctcaaggatcttaccgctg
ttgagatccagttcgatgtaacccactcgtgcacccaactgatcttcagcatcttttactttcaccagcgtttctgggtgagcaaaaacaggaaggcaa
aatgccgcaaaaaagggaataagggcgacacggaaatgttgaatactcatactcttcctttttcaatattattgaagcatttatcagggttattgtctc
atgagcggatacatatttgaatgtatttagaaaaataaacaaataggggttccgcgcacatttccccgaaaagtgccacctgacgtctaagaaaccatt
attatcatgacattaacctataaaaataggcgtatcacgaggccctttcgtctcgcgcgtttcggtgatgacggtgaaaacctctgacacatgcagctc
ccggagacggtcacagcttgtctgtaagcggatgccgggagcagacaagcccgtcagggcgcgtcagcgggtgttggcgggtgtcggggctggcttaac
tatgcggcatcagagcagattgtactgagagtgcaccatatgcggtgtgaaataccgcacagatgcgtaaggagaaaataccgcatcaggcgccatt
cgccattcaggctgcgcaactgttgggaagggcgatcggtgcgggcctcttcgctattacgccagctggcgaaagggggatgtgctgcaaggcgat
taagttgggtaacgccagggttttcccagtcacgacgttgtaaaacgacggcgcaaggaatggtgcatgcaaggagatggcgcccaacagtcccc
cggccacggggcctgccaccatacccacgccgaaacaagcgctcatgagcccgaagtggcgagcccgatcttccccatcggtgatgtcggcgat
ataggcgccagcaaccgcacctgtggcgccggtgatgccggccacgatgcgtccggcgtagaggcgattagtccaatttgttaaagacaggatat
cagtggtccaggctctagttttgactcaacaatatcaccagctgaagcctatagagtacgagccatagataaaataaaagattttatttagtctccaga
aaaagggggg
SEQ ID NO: 8-Mouse a806 CAR
pELI0090 MSGV-SP 806 VHVL-28Z-T2A mCHERRY AII ITAMs intact Sequences MSGV
aatgaaagaccccacctgtaggtttggcaagctagcttaagtaacgccattttgcaaggcatggaaaatacataactgagaatagagaagttcaga
tcaaggttaggaacagagagacagcagaatatgggccaaacaggatatctgtggtaagcagttcctgccccggctcagggccaagaacagatg
gtccccagatgcggtcccgccctcagcagtttctagagaaccatcagatgtttccagggtgccccaaggacctgaaatgaccctgtgccttatttgaa
ctaaccaatcagttcgcttctcgcttctgttcgcgcgcttctgctccccgagctcaataaaagagcccacaacccctcactcggcgcgccagtcctccg
atagactgcgtcgcccgggtacccgtattcccaataaagcctcttgctgtttgcatccgaatcgtggactcgctgatccttgggagggtctcctcagat
tgattgactgcccacctcgggggtctttcatttggaggttccaccgagatttggagacccctgcccagggaccaccgacccccccgccgggaggtaag
ctggccagcggtcgtttcgtgtctgtctctgtctttgtgcgtgtttgtgccggcatctaatgtttgcgcctgcgtctgtactagttagctaactagctc
tgtatctggcggacccgtggtggaactgacgagttcggaacacccggccgcaaccctgggagacgtcccagggacttcgggggccgtttttgtggcccg
acctgagtcctaaaatcccgatcgtttaggactctttggtgcaccccccttagaggagggatatgtggttctggtaggagacgagaacctaaaacagtt
cccgcctccgtctgaatttttgctttcggtttgggaccgaagccgcgccgcgcgtcttgtctgctgcagcatcgttctgtgttgtctctgtctgactgt
gtttctgtatttgtctgaaaatatgggcccgggctagcctgttaccactcccttaagtttgaccttaggtcactggaaagatgtcgagcggatcgctca
caaccagtcggtagatgtcaagaagagacgttgggttaccttctgctctgcagaatggccaacctttaacgtcggatggccgcgagacggcacctttaa
ccgagacctcatcacccaggttaagatcaaggtcttttcacctggcccgcatggacacccagaccaggtcccctacatcgtgacctgggaagccttggc
ttttgacccccctccctgggtcaagccctttgtacaccctaagcctccgcctcctcttcctccatccgccccgtctctcccccttgaacctcctcgttc
gaccccgcctcgatcctccctttatccagccctcactccttctctaggcgcccccatatggccatatgagatcttatatggggcacccccgccccttgt
aaacttccctgaccctgacatgacaagagttactaacagcccctctctccaagctcacttacaggctctctacttagtccagcacgaagtctggagacc
tctggcggcagcctaccaagaacaactggaccgaccggtggtacctcacccttaccgagtcggcgacacagtgtgggtccgccgacaccagactaagaa
cctagaacctcgctggaaaggaccttacacagtcctgctgaccacccccaccgccctcaaagtagacggcatcgcagcttggatacacgccgcccacg
tgaaggctgccgaccccgggggtggaccatcctctagccctcgagaagcttgccaccATGGCATTGCCCGTGACCGCCCTGCT
GCTGCCACTGGCCTTGTTGCTCCACGCCGCGCGGCCAGAACAGAAGCTGATCAGCGAGGAGGATC
TGGATGTCCAGCTGCAAGAGTCTGGCCCTAGCCTGGTCAAGCCTAGCCAGAGCCTGAGCCTGACAT
GTACCGTGACCGGCTACAGCATCACCAGCGACTTCGCCTGGAACTGGATCAGACAGTTCCCCGGCA
ACAAGCTGGAATGGATGGGCTACATCAGCTACAGCGGCAACACCCGGTACAACCCCAGCCTGAAGT
CCCGGATCTCCATCACCAGAGACACCAGCAAGAACCAGTTCTTCCTGCAGCTGAACAGCGTGACCA
TCGAGGACACCGCCACCTACTACTGTGTGACAGCCGGCAGAGGCTTCCCTTATTGGGGACAGGGAA
CCCTGGTCACAGTGTCTGCTGGTGGCGGAGGATCTGGCGGAGGCGGATCTTCTGGCGGTGGCTCT
GATATCCTGATGACACAGAGCCCCAGCAGCATGTCTGTGTCCCTGGGCGATACCGTGTCCATCACC
TGTCACAGCAGCCAGGACATCAACAGCAACATCGGCTGGCTGCAGCAGAGGCCTGGCAAGTCTTTT
AAGGGCCTGATCTACCACGGCACCAACCTGGATGATGAGGTGCCCAGCAGATTTTCCGGCTCTGGA
AGCGGAGCCGACTACTCCCTGACAATCAGCAGCCTGGAAAGCGAGGACTTCGCCGATTACTACTGC
GTGCAGTACGCCCAGTTTCCTTGGACCTTTGGAGGCGGCACAAAGCTGGAAATCAAGCGGatcgagttc
atgtacccccctccctacctggacaacgagagaagcaacggcaccatcatccacatcaaagaaaagcacctgtgccacacccagagcagccc
caagctgttctgggccctggtggtggtggccggcgtgctgttctgttacggcctgctggtcacagtggccctgtgcgtgatctggaccaacagcagaa
gaaacagaggcggccagagcgactacatgaacatgacccccagaaggccaggcctgaccagaaagccctaccagccctacgcccctgccag
agacttcgccgcctacagacccagagccaagttcagcagatccgccgagacagccgccaacctgcaggatcccaaccagctgtacaacgagct
gaacctgggcagacgggaggaatacgacgtgctggaaaagaagagagccagggaccccgagatgggcggcaagcagcagagaagaaga
aaccctcaggaaggcgtctacaacgccctgcagaaagacaagatggccgaggcctacagcgagatcggcaccaagggcgagagaagaagg
ggcaagggccacgatggcctgtaccagggcctgtccaccgccaccaaggacacctacgacgccctgcacatgcagaccctggcccccagaT
CCGGAGGCGGCGGAGAGGGCAGAGGAAGTCTTCTAACATGCGGTGACGTGGAGGAGAATCCCGG
CCCTAGGATGGTCTCCAAAGGGGAGGAAGACAACATGGCTATCATCAAAGAATTTATGAGATTTAAG
GTCCATATGGAAGGCTCAGTGAATGGTCACGAATTCGAGATCGAAGGCGAAGGAGAGGGTCGGCCT
TATGAGGGGACACAGACAGCTAAACTTAAGGTTACAAAAGGTGGACCATTGCCCTTTGCCTGGGACA
TCCTCAGTCCACAGTTTATGTATGGGAGCAAAGCCTATGTTAAGCATCCCGCAGACATCCCTGATTA
CCTTAAGCTTTCATTTCCCGAAGGTTTCAAGTGGGAGAGGGTAATGAACTTTGAAGACGGGGGGGTT
GTTACCGTTACACAGGATAGCAGTCTTCAAGATGGCGAATTTATCTATAAGGTGAAACTGCGGGGCA
CCAACTTTCCCAGCGATGGGCCTGTGATGCAAAAGAAAACAATGGGATGGGAAGCAAGTAGCGAAA
GGATGTACCCAGAAGATGGTGCCCTTAAGGGAGAGATCAAGCAACGGCTTAAACTGAAAGATGGTG
GACACTACGATGCAGAAGTCAAAACAACTTACAAAGCCAAAAAGCCCGTTCAACTCCCAGGTGCCTA
CAACGTTAATATTAAGCTGGACATTACAAGTCATAATGAAGATTACACTATAGTTGAGCAGTACGAGC
GGGCCGAGGGCAGGCACAGCACAGGTGGTATGGACGAACTGTATAAGTAAgtcgacggtaccgcgggcccg
ggatccgataaaataaaagattttatttagtctccagaaaaaggggggaatgaaagaccccacctgtaggtttggcaagctagcttaagtaacgcc
attttgcaaggcatggaaaatacataactgagaatagagaagttcagatcaaggttaggaacagagagacagcagaatatgggccaaacagga
tatctgtggtaagcagttcctgccccggctcagggccaagaacagatggtccccagatgcggtcccgccctcagcagtttctagagaaccatcagat
gtttccagggtgccccaaggacctgaaatgaccctgtgccttatttgaactaaccaatcagttcgcttctcgcttctgttcgcgcgcttctgctccccg
agctcaataaaagagcccacaacccctcactcggcgcgccagtcctccgatagactgcgtcgcccgggtacccgtgtatccaataaaccctcttgcagt
tgcatccgacttgtggtctcgctgttccttgggagggtctcctctgagtgattgactacccgtcagcgggggtctttcatgggtaacagtttcttgaag
ttggagaacaacattctgagggtaggagtcgaatattaagtaatcctgactcaattagccactgttttgaatccacatactccaatactcctgaaatcc
atcgatggagttcattatggacagcgcagaaagagctggggagaattgtgaaattgttatccgctcacaattccacacaacatacgagccggaagcataa
agtgtaaagcctggggtgcctaatgagtgagctaactcacattaattgcgttgcgctcactgcccgctttccagtcgggaaacctgtcgtgccagctgc
attaatgaatcggccaacgcgcggggagaggcggtttgcgtattgggcgctcttccgcttcctcgctcactgactcgctgcgctcggtcgttcggctgc
ggcgagcggtatcagctcactcaaaggcggtaatacggttatccacagaatcaggggataacgcaggaaagaacatgtgagcaaaaggccag
caaaaggccaggaaccgtaaaaaggccgcgttgctggcgtttttccataggctccgcccccctgacgagcatcacaaaaatcgacgctcaagtca
gaggtggcgaaacccgacaggactataaagataccaggcgtttccccctggaagctccctcgtgcgctctcctgttccgaccctgccgcttaccgga
tacctgtccgcctttctcccttcgggaagcgtggcgctttctcatagctcacgctgtaggtatctcagttcggtgtaggtcgttcgctccaagctgggc
tgtgtgcacgaaccccccgttcagcccgaccgctgcgccttatccggtaactatcgtcttgagtccaacccggtaagacacgacttatcgccactggca
gcagccactggtaacaggattagcagagcgaggtatgtaggcggtgctacagagttcttgaagtggtggcctaactacggctacactagaagaacag
tatttggtatctgcgctctgctgaagccagttaccttcggaaaaagagttggtagctcttgatccggcaaacaaaccaccgctggtagcggtggttttt
ttgtttgcaagcagcagattacgcgcagaaaaaaaggatctcaagaagatcctttgatcttttctacggggtctgacgctcagtggaacgaaaactcac
gttaagggattttggtcatgagattatcaaaaaggatcttcacctagatccttttaaattaaaaatgaagttttaaatcaatctaaagtatatatgagt
aaacttggtctgacagttaccaatgcttaatcagtgaggcacctatctcagcgatctgtctatttcgttcatccatagttgcctgactccccgtcgtgt
agataactacgatacgggagggcttaccatctggccccagtgctgcaatgataccgcgagacccacgctcaccggctccagatttatcagcaataaacc
agccagccggaagggccgagcgcagaagtggtcctgcaactttatccgcctccatccagtctattaattgttgccgggaagctagagtaagtagttcgc
cagttaatagtttgcgcaacgttgttgccattgctacaggcatcgtggtgtcacgctcgtcgtttggtatggcttcattcagctccggttcccaacgat
caaggcgagttacatgatcccccatgttgtgcaaaaaagcggttagctccttcggtcctccgatcgttgtcagaagtaagttggccgcagtgttatcac
tcatggttatggcagcactgcataattctcttactgtcatgccatccgtaagatgcttttctgtgactggtgagtactcaaccaagtcattctgagaat
agtgtatgcggcgaccgagttgctcttgcccggcgtcaatacgggataataccgcgccacatagcagaactttaaaagtgctcatcattggaaaacgtt
cttcggggcgaaaactctcaaggatcttaccgctgttgagatccagttcgatgtaacccactcgtgcacccaactgatcttcagcatcttttactttca
ccagcgtttctgggtgagcaaaaacaggaaggcaaaatgccgcaaaaaagggaataagggcgacacggaaatgttgaatactcatactcttcctttttc
aatattattgaagcatttatcagggttattgtctcatgagcggatacatatttgaatgtatttagaaaaataaacaaataggggttccgcgcacattt
ccccgaaaagtgccacctgacgtctaagaaaccattattatcatgacattaacctataaaaataggcgtatcacgaggccctttcgtctcgcgcgttt
cggtgatgacggtgaaaacctctgacacatgcagctcccggagacggtcacagcttgtctgtaagcggatgccgggagcagacaagcccgtcagggcg
cgtcagcgggtgttggcgggtgtcggggctggcttaactatgcggcatcagagcagattgtactgagagtgcaccatatgcggtgtgaaataccgcac
agatgcgtaaggagaaaataccgcatcaggcgccattcgccattcaggctgcgcaactgttgggaagggcgatcggtgcgggcctcttcgctattacg
ccagctggcgaaagggggatgtgctgcaaggcgattaagttgggtaacgccagggttttcccagtcacgacgttgtaaaacgacggcgcaaggaatg
gtgcatgcaaggagatggcgcccaacagtcccccggccacggggcctgccaccatacccacgccgaaacaagcgctcatgagcccgaagtgg
cgagcccgatcttccccatcggtgatgtcggcgatataggcgccagcaaccgcacctgtggcgccggtgatgccggccacgatgcgtccggcgta
gaggcgattagtccaatttgttaaagacaggatatcagtggtccaggctctagttttgactcaacaatatcaccagctgaagcctatagagtacgagc
catagataaaataaaagattttatttagtctccagaaaaagggggg
SEQ ID NO: 9-Mouse aEGFRviii CAR
pELI0039 MSGV-EGFRVIII-28Z-T2A mCHERRY AII ITAMs intact Sequences
aatgaaagaccccacctgtaggtttggcaagctagcttaagtaacgccattttgcaaggcatggaaaatacataactgagaatagagaagttcaga
tcaaggttaggaacagagagacagcagaatatgggccaaacaggatatctgtggtaagcagttcctgccccggctcagggccaagaacagatg
gtccccagatgcggtcccgccctcagcagtttctagagaaccatcagatgtttccagggtgccccaaggacctgaaatgaccctgtgccttatttgaa
ctaaccaatcagttcgcttctcgcttctgttcgcgcgcttctgctccccgagctcaataaaagagcccacaacccctcactcggcgcgccagtcctccg
atagactgcgtcgcccgggtacccgtattcccaataaagcctcttgctgtttgcatccgaatcgtggactcgctgatccttgggagggtctcctcagat
tgattgactgcccacctcgggggtctttcatttggaggttccaccgagatttggagacccctgcccagggaccaccgacccccccgccgggaggtaag
ctggccagcggtcgtttcgtgtctgtctctgtctttgtgcgtgtttgtgccggcatctaatgtttgcgcctgcgtctgtactagttagctaactagct
ctgtatctggcggacccgtggtggaactgacgagttcggaacacccggccgcaaccctgggagacgtcccagggacttcgggggccgtttttgtggccc
gacctgagtcctaaaatcccgatcgtttaggactctttggtgcaccccccttagaggagggatatgtggttctggtaggagacgagaacctaaaacagt
tcccgcctccgtctgaatttttgctttcggtttgggaccgaagccgcgccgcgcgtcttgtctgctgcagcatcgttctgtgttgtctctgtctgactg
tgtttctgtatttgtctgaaaatatgggcccgggctagcctgttaccactcccttaagtttgaccttaggtcactggaaagatgtcgagcggatcgctc
acaaccagtcggtagatgtcaagaagagacgttgggttaccttctgctctgcagaatggccaacctttaacgtcggatggccgcgagacggcaccttta
accgagacctcatcacccaggttaagatcaaggtcttttcacctggcccgcatggacacccagaccaggtcccctacatcgtgacctgggaagccttgg
cttttgacccccctccctgggtcaagccctttgtacaccctaagcctccgcctcctcttcctccatccgccccgtctctcccccttgaacctcctcgtt
cgaccccgcctcgatcctccctttatccagccctcactccttctctaggcgcccccatatggccatatgagatcttatatggggcacccccgccccttg
taaacttccctgaccctgacatgacaagagttactaacagcccctctctccaagctcacttacaggctctctacttagtccagcacgaagtctggagac
ctctggcggcagcctaccaagaacaactggaccgaccggtggtacctcacccttaccgagtcggcgacacagtgtgggtccgccgacaccagactaaga
acctagaacctcgctggaaaggaccttacacagtcctgctgaccacccccaccgccctcaaagtagacggcatcgcagcttggatacacgccgcccacg
tgaaggctgccgaccccgggggtggaccatcctctagccctcgagaagcttgccaccatgggtgtccctacccagctcctgggactgctcctgctgt
ggatcaccgacgccatctgcGAGGTGCAGGTGTTGGAGTCTGGGGGAGGCTTGGTACAGCCTGGGGGGTCC
CTGAGACTCTCCTGTGCAGCCTCTGGATTCACCTTTAGCAGCTATGCCATGAGCTGGGTCCGCCAG
GCTCCAGGGAAGGGGCTGGAGTGGGTCTCGGCTATTAGTGGTAGTGGTGGTAGTACAAACTACGCA
GACTCCGTGAAGGGCCGGTTCACCATCTCCAGAGACAATTCCAAGAACACACTGTATCTGCAAATGA
ACAGCCTGAGAGCCGAGGACACGGCCGTCTATTACTGTGCTGGGAGCAGTGGCTGGTCCGAGTAC
TGGGGCCAGGGAACCCTGGTCACCGTCTCCTCGGGTGGAGGTGGATCAGGTGGAGGTGGATCTGG
TGGAGGTGGATCTGACATCCAGATGACCCAGTCTCCATCCTCCCTGTCTGCATCTGTAGGAGACAGA
GTCACCATCACTTGCCGGGCTAGTCAGGGCATTAGAAATAATTTAGCCTGGTATCAGCAGAAACCAG
GGAAAGCCCCTAAGCGCCTGATCTATGCTGCCTCCAATTTGCAAAGTGGGGTCCCATCAAGGTTCAC
CGGCAGTGGATCTGGGACAGAATTCACTCTCATAGTCAGCAGCCTGCAGCCTGAAGATTTTGCGACT
TATTACTGTCTACAGCATCACAGTTACCCGCTCACTTCCGGCGGAGGGACCAAGGTGGAGATCAAAa
tcgagttcatgtacccccctccctacctggacaacgagagaagcaacggcaccatcatccacatcaaagaaaagcacctgtgccacacccagag
cagccccaagctgttctgggccctggtggtggtggccggcgtgctgttctgttacggcctgctggtcacagtggccctgtgcgtgatctggaccaacag
cagaagaaacagaggcggccagagcgactacatgaacatgacccccagaaggccaggcctgaccagaaagccctaccagccctacgcccc
tgccagagacttcgccgcctacagacccagagccaagttcagcagatccgccgagacagccgccaacctgcaggatcccaaccagctgtacaa
cgagctgaacctgggcagacgggaggaatacgacgtgctggaaaagaagagagccagggaccccgagatgggggcaagcagcagagaa
gaagaaaccctcaggaaggcgtctacaacgccctgcagaaagacaagatggccgaggcctacagcgagatcggcaccaagggcgagagaa
gaaggggcaagggccacgatggcctgtaccagggcctgtccaccgccaccaaggacacctacgacgccctgcacatgcagaccctggccccc
agaTCCGGAGGCGGCGGAGAGGGCAGAGGAAGTCTTCTAACATGCGGTGACGTGGAGGAGAATCC
CGGCCCTAGGATGGTCTCCAAAGGGGAGGAAGACAACATGGCTATCATCAAAGAATTTATGAGATTT
AAGGTCCATATGGAAGGCTCAGTGAATGGTCACGAATTCGAGATCGAAGGCGAAGGAGAGGGTCGG
CCTTATGAGGGGACACAGACAGCTAAACTTAAGGTTACAAAAGGTGGACCATTGCCCTTTGCCTGGG
ACATCCTCAGTCCACAGTTTATGTATGGGAGCAAAGCCTATGTTAAGCATCCCGCAGACATCCCTGA
TTACCTTAAGCTTTCATTTCCCGAAGGTTTCAAGTGGGAGAGGGTAATGAACTTTGAAGACGGGGGG
GTTGTTACCGTTACACAGGATAGCAGTCTTCAAGATGGCGAATTTATCTATAAGGTGAAACTGCGGG
GCACCAACTTTCCCAGCGATGGGCCTGTGATGCAAAAGAAAACAATGGGATGGGAAGCAAGTAGCG
AAAGGATGTACCCAGAAGATGGTGCCCTTAAGGGAGAGATCAAGCAACGGCTTAAACTGAAAGATG
GTGGACACTACGATGCAGAAGTCAAAACAACTTACAAAGCCAAAAAGCCCGTTCAACTCCCAGGTGC
CTACAACGTTAATATTAAGCTGGACATTACAAGTCATAATGAAGATTACACTATAGTTGAGCAGTACG
AGCGGGCCGAGGGCAGGCACAGCACAGGTGGTATGGACGAACTGTATAAGTAACGCTCGACAATC
AACCTCTGGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTCTCCTCCT
TGTATAAATCCTGGTTGCTGTCTCTTTATGAGGAGTTGTGGCCCGTTGTCAGGCAACGTGGCGTGGT
GTGCACTGTGTTTGCTGACGCAACCCCCACTGGTTGGGGCATTGCCACCACCTGTCAGCTCCTTTC
CGGGACTTTCGCTTTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGAAGCTGACGTCCTT
TCCATGGCTGCTCGCCTGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCGCCTGgtcgacggtaccgcgggc
ccgggatccgataaaataaaagattttatttagtctccagaaaaaggggggaatgaaagaccccacctgtaggtttggcaagctagcttaagtaac
gccattttgcaaggcatggaaaatacataactgagaatagagaagttcagatcaaggttaggaacagagagacagcagaatatgggccaaaca
ggatatctgtggtaagcagttcctgccccggctcagggccaagaacagatggtccccagatgcggtcccgccctcagcagtttctagagaaccatc
agatgtttccagggtgccccaaggacctgaaatgaccctgtgccttatttgaactaaccaatcagttcgcttctcgcttctgttcgcgcgcttctgct
ccccgagctcaataaaagagcccacaacccctcactcggcgcgccagtcctccgatagactgcgtcgcccgggtacccgtgtatccaataaaccctcttg
cagttgcatccgacttgtggtctcgctgttccttgggagggtctcctctgagtgattgactacccgtcagcgggggtctttcatgggtaacagtttcttg
aagttggagaacaacattctgagggtaggagtcgaatattaagtaatcctgactcaattagccactgttttgaatccacatactccaatactcctgaaat
ccatcgatggagttcattatggacagcgcagaaagagctggggagaattgtgaaattgttatccgctcacaattccacacaacatacgagccggaagca
taaagtgtaaagcctggggtgcctaatgagtgagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcgctcactgactcgctgcgctcggtcgttcggc
tgcggcgagcggtatcagctcactcaaaggcggtaatacggttatccacagaatcaggggataacgcaggaaagaacatgtgagcaaaaggcc
agcaaaaggccaggaaccgtaaaaaggccgcgttgctggcgtttttccataggctccgcccccctgacgagcatcacaaaaatcgacgctcaagt
cagaggtggcgaaacccgacaggactataaagataccaggcgtttccccctggaagctccctcgtgcgctctcctgttccgaccctgccgcttaccg
gatacctgtccgcctttctcccttcgggaagcgtggcgctttctcatagctcacgctgtaggtatctcagttcggtgtaggtcgttcgctccaagctgg
gctgtgtgcacgaaccccccgttcagcccgaccgctgcgccttatccggtaactatcgtcttgagtccaacccggtaagacacgacttatcgccactgg
cagcagccactggtaacaggattagcagagcgaggtatgtaggcggtgctacagagttcttgaagtggtggcctaactacggctacactagaagaa
cagtatttggtatctgcgctctgctgaagccagttaccttcggaaaaagagttggtagctcttgatccggcaaacaaaccaccgctggtagcggtggtt
tttttgtttgcaagcagcagattacgcgcagaaaaaaaggatctcaagaagatcctttgatcttttctacggggtctgacgctcagtggaacgaaaact
cacgttaagggattttggtcatgagattatcaaaaaggatcttcacctagatccttttaaattaaaaatgaagttttaaatcaatctaaagtatatatg
agtaaacttggtctgacagttaccaatgcttaatcagtgaggcacctatctcagcgatctgtctatttcgttcatccatagttgcctgactccccgtgt
gtagataactacgatacgggagggcttaccatctggccccagtgctgcaatgataccgcgagacccacgctcaccggctccagatttatcagcaataaa
ccagccagccggaagggccgagcgcagaagtggtcctgcaactttatccgcctccatccagtctattaattgttgccgggaagctagagtaagtagttc
gccagttaatagtttgcgcaacgttgttgccattgctacaggcatcgtggtgtcacgctcgtcgtttggtatggcttcattcagctccggttcccaacg
atcaaggcgagttacatgatcccccatgttgtgcaaaaaagcggttagctccttcggtcctccgatcgttgtcagaagtaagttggccgcagtgttatc
actcatggttatggcagcactgcataattctcttactgtcatgccatccgtaagatgcttttctgtgactggtgagtactcaaccaagtcattctgaga
atagtgtatgcggcgaccgagttgctcttgcccggcgtcaatacgggataataccgcgccacatagcagaactttaaaagtgctcatcattggaaaacg
ttcttcggggcgaaaactctcaaggatcttaccgctgttgagatccagttcgatgtaacccactcgtgcacccaactgatcttcagcatcttttacttt
caccagcgtttctgggtgagcaaaaacaggaaggcaaaatgccgcaaaaaagggaataagggcgacacggaaatgttgaatactcatactcttcctttt
tcaatattattgaagcatttatcagggttattgtctcatgagcggatacatatttgaatgtatttagaaaaataaacaaataggggttccgcgcacatt
tccccgaaaagtgccacctgacgtctaagaaaccattattatcatgacattaacctataaaaataggcgtatcacgaggccctttcgtctcgcgcgtt
tcggtgatgacggtgaaaacctctgacacatgcagctcccggagacggtcacagcttgtctgtaagcggatgccgggagcagacaagcccgtcagggc
gcgtcagcgggtgttggcgggtgtcggggctggcttaactatgcggcatcagagcagattgtactgagagtgcaccatatgcggtgtgaaataccgcac
agatgcgtaaggagaaaataccgcatcaggcgccattcgccattcaggctgcgcaactgttgggaagggcgatcggtgcgggcctcttcgctattacg
ccagctggcgaaagggggatgtgctgcaaggcgattaagttgggtaacgccagggttttcccagtcacgacgttgtaaaacgacggcgcaagga
atggtgcatgcaaggagatggcgcccaacagtcccccggccacggggcctgccaccatacccacgccgaaacaagcgctcatgagcccgaag
tggcgagcccgatcttccccatcggtgatgtcggcgatataggcgccagcaaccgcacctgtggcgccggtgatgccggccacgatgcgtccggc
gtagaggcgattagtccaatttgttaaagacaggatatcagtggtccaggctctagttttgactcaacaatatcaccagctgaagcctatagagtacga
gccatagataaaataaaagattttatttagtctccagaaaaagggggg
SEQ ID NO: 10 Retroviral transduction of commercial B16-F10 cell line expressing extracellular
domain of EGFRviii with GFP and fLUC
tgaaagaccccacctgtaggtttggcaagctagcttaagtaacgccattttgcaaggcatggaaaatacataactgagaatagagaagttcagatc
aaggttaggaacagagagacagcagaatatgggccaaacaggatatctgtggtaagcagttcctgccccggctcagggccaagaacagatggt
ccccagatgcggtcccgccctcagcagtttctagagaaccatcagatgtttccagggtgccccaaggacctgaaatgaccctgtgccttatttgaact
aaccaatcagttcgcttctcgcttctgttcgcgcgcttctgctccccgagctcaataaaagagcccacaacccctcactcggcgcgccagtcctccgat
agactgcgtcgcccgggtacccgtattcccaataaagcctcttgctgtttgcatccgaatcgtggactcgctgatccttgggagggtctcctcagattg
attgactgcccacctcgggggtctttcatttggaggttccaccgagatttggagacccctgcccagggaccaccgacccccccgccgggaggtaagct
ggccagcggtcgtttcgtgtctgtctctgtctttgtgcgtgtttgtgccggcatctaatgtttgcgcctgcgtctgtactagttagctaactagctctg
tatctggcggacccgtggtggaactgacgagttctgaacacccggccgcaaccctgggagacgtcccagggactttgggggccgtttttgtggcccgac
ctgaggaagggagtcgatgtggaatccgaccccgtcaggatatgtggttctggtaggagacgagaacctaaaacagttcccgcctccgtctgaattttt
gctttcggtttggaaccgaagccgcgcgtcttgtctgctgcagcgctgcagcatcgttctgtgttgtctctgtctgactgtgtttctgtatttgtctga
aaattagggccagactgttaccactcccttaagtttgaccttaggtcactggaaagatgtcgagcggatcgctcacaaccagtcggtagatgtcaagaa
gagacgttgggttaccttctgctctgcagaatggccaacctttaacgtcggatggccgcgagacggcacctttaaccgagacctcatcacccaggtta
agatcaaggtcttttcacctggcccgcatggacacccagaccaggtcccctacatcgtgacctgggaagccttggcttttgacccccctccctgggtca
agccctttgtacaccctaagcctccgcctcctcttcctccatccgccccgtctctcccccttgaacctcctcgttcgaccccgcctcgatcctcccttt
atccagccctcactccttctctaggcgccggaattagatctctcgaggttctgagagatctaccGCCGCCACCatgcgaccctcagggaccgcgagaa
ccacactgctggtgttgctgaccgcgctctgcgccgcaggtggggcgttggaggaaaagaaaggaaactacgtggtgacagatcatggctcatgtgtc
cgagcctgtgggcctgactactacgaagtggaagaagatggcatccgcaagtgtaaaaaatgtgatgggccctgtcgcaaagtttgtaatggcataggc
attggtgaatttaaagacacactctccataaatgctacaaacatcaaacacttcaaatactgcactgccatcagcggggaccttcacatcctgccagt
ggcctttaagggggattctttcacgcgcactcctcctctagacccacgagaactagaaattctaaaaaccgtaaaggaaataacaggctttttgctgat
tcaggcttggcctgataactggactgacctccatgctttcgagaacctagaaataatacgtggcagaacaaagcaacatggtcagttttctttggcggt
cgttggcctgaacatcacatcactggggctgcgttccctcaaggagatcagtgatggggatgtgatcatttctggaaaccgaaatttgtgctacgcaa
acacaataaactggaaaaaactcttcgggacacccaatcagaaaaccaaaatcatgaacaacagagctgagaaagactgcaaggccgtgaa
ccacgtctgcaatcctttatgctcctcggaaggctgctggggccctgagcccagggactgtgtctcctgccagaatgtgagcagaggcagggagtg
cgtggagaaatgcaacatcctggagggggaaccaagggagtttgtggaaaattctgaatgcatccagtgccatccagaatgtctgccccaggcca
tgaacatcacctgtacaggcaggggaccagacaactgcatccagtgtgcccactacattgatggcccacactgtgtcaagacctgcccagctggc
atcatgggagagaacaacactctggtctggaagtatgcagatgccaataatgtctgccacctatgccacgccaactgtacctatggatgtgctgggc
caggtcttcaaggatgtgaagtgtggccatctgggccaaagataccatctattgccactgggattgtgggtggcctcctcttcatagtggtggtggccc
ttgggattggcctattcatgcgaagacgtcacattgttcgaaagcgtacactacgccgcctgcttcaagagagagagctcgtggaacctctcacaccc
agcggagaagctccaaaccaagcccacttgaggatattaaaggaaacagaattcaaaaagatcaaagttctgggttcgggagcatttggcacag
tgtataagggtctctggatcccagaaggtgagaaagtaaaaatcccggtggccatcaaggagttaagagaagccacatctccaaaagccaacaa
agaaatccttgacgaagcctatgtgatggctagtgtggacaaccctcatgtatgccgcctcctgggcatctgtctgacctccactgtccagctcattac
acagctcatgccctacggttgcctcctggactacgtccgagaacacaaggacaacattggctcccagtacctcctcaactggtgtgtgcagattgcaa
agggcatgaactacctggaagatcggcgtttggtgcaccgtgacttggcagccaggaatgtactggtgaagacaccacagcatgtcaagatcaca
gattttgggctggccaaactgcttggtgctgaagagaaagaatatcatgccgaggggggcaaagtgcctatcaagtggatggctttggaatcaatttt
acaccgaatttatacacaccaaagtgatgtctggagctatggtgtcactgtgtgggaactgatgacctttgggtccaagccttatgatggaatcccagc
aagtgacatctcatccatcctagagaaaggagagcgccttccacagccacctTCCGGAGGCGGCGGAGAGGGCAGAGGAA
GTCTTCTAACATGCGGTGACGTGGAGGAGAATCCCGGCCCTAGGatggtgagcaagggcgaggagctgttcaccg
gggtggtgcccatcctggtcgagctggacggcgacgtaaacggccacaagttcagcgtgtccggcgagggcgagggcgatgccacctacggca
agctgaccctgaagttcatctgcaccaccggcaagctgcccgtgccctggcccaccctcgtgaccaccctgacctacggcgtgcagtgcttcagcc
gctaccccgaccacatgaagcagcacgacttcttcaagtccgccatgcccgaaggctacgtccaggagcgcaccatcttcttcaaggacgacggc
aactacaagacccgcgccgaggtgaagttcgagggcgacaccctggtgaaccgcatcgagctgaagggcatcgacttcaaggaggacggcaa
catcctggggcacaagctggagtacaactacaacagccacaacgtctatatcatggccgacaagcagaagaacggcatcaaggtgaacttcaa
gatccgccacaacatcgaggacggcagcgtgcagctcgccgaccactaccagcagaacacccccatcggcgacggccccgtgctgctgcccg
acaaccactacctgagcacccagtccgccctgagcaaagaccccaacgagaagcgcgatcacatggtcctgctggagttcgtgaccgccgccg
ggatcactctcggcatggacgagctgtacaagTCCGGAGCCACCAACTTCAGCCTGCTGAAGCAGGCCGGCGACG
TGGAGGAGAACCCTGGCCCTCCTAGGatggtgaagcgtgagaaaaatgtcatctatggccctgagcctctccatcctttggaggat
ttgactgccggcgaaatgctgtttcgtgctctccgcaagcactctcatttgcctcaagccttggtcgatgtggtcggcgatgaatctttgagctacaag
gagttttttgaggcaaccgtcttgctggctcagtccctccacaattgtggctacaagatgaacgacgtcgttagtatctgtgctgaaaacaatacccgt
ttcttcattccagtcatcgccgcatggtatatcggtatgatcgtggctccagtcaacgagagctacattcccgacgaactgtgtaaagtcatgggtat
ctctaagccacagattgtcttcaccactaagaatattctgaacaaagtcctggaagtccaaagccgcaccaactttattaagcgtatcatcatcttgga
cactgtggagaatattcacggttgcgaatctttgcctaatttcatctctcgctattcagacggcaacatcgcaaactttaaaccactccacttcgaccc
tgtggaacaagttgcagccattctgtgtagcagcggtactactggactcccaaagggagtcatgcagacccatcaaaacatttgcgtgcgtctgatcc
atgctctcgatccacgcgtgggcactcagctgattcctggtgtcaccgtcttggtctacttgcctttcttccatgctttcggctttagcattactttgg
gttactttatggtcggtctccgcgtgattatgttccgccgttttgatcaggaggctttcttgaaagccatccaagattatgaagtccgcagtgtcatca
acgtgcctagcgtgatcctgtttttgtctaagagcccactcgtggacaagtacgacttgtcttcactgcgtgaattgtgttgcggtgccgctccactgg
ctaaggaggtcgctgaagtggccgccaaacgcttgaatcttccagggattcgttgtggcttcggcctcaccgaatctaccagcgctaacattcactctc
tcggggatgagtttaagagcggctctttgggccgtgtcactccactcatggctgctaagatcgctgatcgcgaaactggtaaggctttgggcccgaacc
aagtgggcgagctgtgtatcaaaggccctatggtgagcaagggttatgtcaataacgttgaagctaccaaggaggccatcgacgacgacggctggttgc
attctggtgattttggatattacgacgaagatgagcatttttacgtcgtggatcgttacaaggagctgatcaaatacaagggtagccaggttgctccag
ctgagttggaggagattctgttgaaaaatccatgcattcgcgatgtcgctgtggtcggcattcctgatctggaggccggcgaactgccttctgctttc
gttgtcaagcagcctggtaaagaaattaccgccaaagaagtgtatgattacctggctgaacgtgtgagccatactaagtacttgcgtggcggcgtgcgt
tttgttgactccatccctcgtaacgtaacaggcaaaattacccgcaaggagctgttgaaacaattgttggagaaggccggcggtTAACGCTCGACAAT
CAACCTCTGGATTACAAAATTTGTGAAAGATTGACTGGTATTCTTAACTATGTTGCTCCTTTTACGCTATGTGGAT
ACGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCA
CTGTGTTTGCTGACGCAACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAAGCTGACGTCCTTTCCAT
GGCTGCTCGCCTGTGTTGCCACCTGGATTCTGCGCGGGACGTCCTTCTGCTACGTCCCTTCGGCCC
TCAATCCAGCGGACCTTCCTTCCCGCGGCCTGCTGCCGGCTCTGCGGCCTCTTCCGCGTCTTCGCC
TTCGCCCTCAGACGAGTCGGATCTCCCTTTGGGCCGCCTCCCCGCCTGctcgagctcagaacgaattcgataaa
ataaaagattttatttagtctccagaaaaaggggggaatgaaagaccccacctgtaggtttggcaagctagcttaagtaacgccattttgcaaggcat
ggaaaatacataactgagaatagagaagttcagatcaaggttaggaacagagagacagcagaatatgggccaaacaggatatctgtggtaagc
agttcctgccccggctcagggccaagaacagatggtccccagatgcggtcccgccctcagcagtttctagagaaccatcagatgtttccagggtgc
cccaaggacctgaaatgaccctgtgccttatttgaactaaccaatcagttcgcttctcgcttctgttcgcgcgcttctgctccccgagctcaataaaag
agcccacaacccctcactcggcgcgccagtcctccgatagactgcgtcgcccgggtacccgtgtatccaataaaccctcttgcagttgcatccgacttg
tggtctcgctgttccttgggagggtctcctctgagtgattgactacccgtcagcgggggtctttcatgggtaacagtttcttgaagttggagaacaac
attctgagggtaggagtcgaatattaagtaatcctgactcaattagccactgttttgaatccacatactccaatactcctgaaatagttcattatggac
agcgcagaaagagctggggagaattgtgaaattgttatccgctcacaattccacacaacatacgagccggaagcataaagtgtaaagcctggggtgccta
atgagtgagctaactcacattaattgcgttgcgctcactgcccgctttccagtcgggaaacctgtcgtgccagctgcattaatgaatcggccaacgcgc
ggggagaggcggtttgcgtattgggcgctcttccgcttcctcgctcactgactcgctgcgctcggtcgttcggctgcggcgagcggtatcagctcactc
aaaggcggtaatacggttatccacagaatcaggggataacgcaggaaagaacatgtgagcaaaaggccagcaaaaggccaggaaccgtaa
aaaggccgcgttgctggcgtttttccataggctccgcccccctgacgagcatcacaaaaatcgacgctcaagtcagaggtggcgaaacccgacag
gactataaagataccaggcgtttccccctggaagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctccaagctgggctgtgtgcacgaaccccccgttc
agcccgaccgctgcgccttatccggtaactatcgtcttgagtccaacccggtaagacacgacttatcgccactggcagcagccactggtaacaggatta
gcagagcgaggtatgtaggcggtgctacagagttcttgaagtggtggcctaactacggctacactagaagaacagtatttggtatctgcgctctgctga
agccagttaccttcggaaaaagagttggtagctcttgatccggcaaacaaaccaccgctggtagcggtggtttttttgtttgcaagcagcagattacgc
gcagaaaaaaaggatctcaagaagatcctttgatcttttctacggggtctgacgctcagtggaacgaaaactcacgttaagggattttggtcatgaga
ttatcaaaaaggatcttcacctagatccttttaaattaaaaatgaagttttaaatcaatctaaagtatatatgagtaaacttggtctgacagttaccaa
tgcttaatcagtgaggcacctatctcagcgatctgtctatttcgttcatccatagttgcctgactccccgtcgtgtagataactacgatacgggagggc
ttaccatctggccccagtgctgcaatgataccgcgagacccacgctcaccggctccagatttatcagcaataaaccagccagccggaagggccgagcgc
agaagtggtcctgcaactttatccgcctccatccagtctattaattgttgccgggaagctagagtaagtagttcgccagttaatagtttgcgcaacgt
tgttgccattgctacaggcatcgtggtgtcacgctcgtcgtttggtatggcttcattcagctccggttcccaacgatcaaggcgagttacatgatcccc
catgttgtgcaaaaaagcggttagctccttcggtcctccgatcgttgtcagaagtaagttggccgcagtgttatcactcatggttatggcagcactgca
taattctcttactgtcatgccatccgtaagatgcttttctgtgactggtgagtactcaaccaagtcattctgagaatagtgtatgcggcgaccgagttg
ctcttgcccggcgtcaatacgggataataccgcgccacatagcagaactttaaaagtgctcatcattggaaaacgttcttcggggcgaaaactctcaag
gatcttaccgctgttgagatccagttcgatgtaacccactcgtgcacccaactgatcttcagcatcttttactttcaccagcgtttctgggtgagcaaa
aacaggaaggcaaaatgccgcaaaaaagggaataagggcgacacggaaatgttgaatactcatactcttcctttttcaatattattgaagcatttatca
gggttattgtctcatgagcggatacatatttgaatgtatttagaaaaataaacaaataggggttccgcgcacatttccccgaaaagtgccacctgacgt
ctaagaaaccattattatcatgacattaacctataaaaataggcgtatcacgaggccctttcgtctcgcgcgtttcggtgatgacggtgaaaacctctg
acacatgcagctcccggagacggtcacagcttgtctgtaagcggatgccgggagcagacaagcccgtcagggcgcgtcagcgggtgttggcgggtgtc
ggggctggcttaactatgcggcatcagagcagattgtactgagagtgcaccatatgcggtgtgaaataccgcacagatgcgtaaggagaaaataccgca
tcaggcgccattcgccattcaggctgcgcaactgttgggaagggcgatcggtgcgggcctcttcgctattacgccagctggcgaaagggggatgtgctg
caaggcgattaagttgggtaacgccagggttttcccagtcacgacgttgtaaaacgacggcgcaaggaatggtgcatgcaaggagatggcgccc
aacagtcccccggccacggggcctgccaccatacccacgccgaaacaagcgctcatgagcccgaagtggcgagcccgatcttccccatcggtg
atgtcggcgatataggcgccagcaaccgcacctgtggcgccggtgatgccggccacgatgcgtccggcgtagaggcgattagtccaatttgttaaa
gacaggatatcagtggtccaggctctagttttgactcaacaatatcaccagctgaagcctatagagtacgagccatagataaaataaaagattttattt
agtctccagaaaaaggggggaa
SEQ ID NO: 11-human EGFR protein sequence (amphiphile conjugate peptide sequence in bold)
DLSFLKTIQEVAGYVLIALNTVERIPLENLQIIRGNMYYENSYALAVLSNYDANKTGLKELPMRNLQEILHGA
VRFSNNPALCNVESIQWRDIVSSDFLSNMSMDFQNHLGSCQKCDPSCPNGSCWGAGEENCQKLTKIICA
QQCSGRCRGKSPSDCCHNQCAAGCTGPRESDCLVCRKFRDEATCKDTCPPLMLYNPTTYQMDVNPEG
KYSFGATCVKKCPRNYVVTDHGSCVRACGADSYEMEEDGVRKCKKCEGPCRKVCNGIGIGEFKDSLSI
NATNIKHFKNCTSISGDLHILPVAFRGDSFTHTPPLDPQELDILKTVKEITGFLLIQAWPENRTDLHAFENLEI
IRGRTKQHGQFSLAVVSLNITSLGLRSLKEISDGDVIISGNKNLCYANTINWKKLFGTSGQKTKIISNRGENS
CKATGQVCHALCSPEGCWGPEPRDCVSCRNVSRGRECVDKCNLLEGEPREFVENSECIQCHPECLPQ
AMNITCTGRGPDNCIQCAHYIDGPHCVKTCPAGVMGENNTLVWKYADAGHVCHLCHPNCTYGCTGPGL
EGCPTNGPKIPSIATGMVGALLLLLVVALGIGLFMRRRHIVRKRTLRRLLQERELVEPLTPSGEAPNQALLR
ILKETEFKKIKVLGSGAFGTVYKGLWIPEGEKVKIPVAIKELREATSPKANKEILDEAYVMASVDNPHVCRLL
GICLTSTVQLITQLMPFGCLLDYVREHKDNIGSQYLLNWCVQIAKGMNYLEDRRLVHRDLAARNVLVKTP
QHVKITDFGLAKLLGAEEKEYHAEGGKVPIKWMALESILHRIYTHQSDVWSYGVTVWELMTFGSKPYDGI
PASEISSILEKGERLPQPPICTIDVYMIMVKCWMIDADSRPKFRELIIEFSKMARDPQRYLVIQGDERMHLP
SPTDSNFYRALMDEEDMDDVVDADEYLIPQQGFFSSPSTSRTPLLSSLSATSNNSTVACIDRNGLQSCPI
KEDSFLQRYSSDPTGALTEDSIDDTFLPVPEYINQSVPKRPAGSVQNPVYHNQPLNPAPSRDPHYQDPH
STAVGNPEYLNTVQPTCVNSTFDSPAHWAQKGSHQISLDNPDYQQDFFPKEAKPNGIFKGSTAENAEYL
RVAPQSSEFIGA
SEQ ID NO:12-CpG oligonucleotide
TCGTCGTTTTGTCGTTTTGTCGTT

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An amphiphilic ligand conjugate, or a pharmaceutically acceptable salt thereof, comprising a lipid, an EGFR peptide, and, optionally, a linker.

2. The amphiphilic ligand conjugate, or pharmaceutically acceptable salt thereof, of claim 1, wherein the EGFR peptide consists of 7 to 40 amino acids.

3. The amphiphilic ligand conjugate, or pharmaceutically acceptable salt thereof, of claim 1, wherein the EGFR peptide is a binding ligand for an EGFR-specific antibody.

4. The amphiphilic ligand conjugate, or pharmaceutically acceptable salt thereof, of claim 3, wherein the EGFR-specific antibody is antibody 806.

5. The amphiphilic ligand conjugate, or pharmaceutically acceptable salt thereof, of claim 1, wherein the EGFR peptide comprises an amino acid sequence of SEQ ID NO: 1, 2, or 5.

6. The amphiphilic ligand conjugate, or pharmaceutically acceptable salt thereof, of claim 1, wherein the EGFR peptide is a cyclic peptide.

7. The amphiphilic ligand conjugate, or pharmaceutically acceptable salt thereof, of claim 5, wherein the peptide comprises an amino acid sequence identical to SEQ ID NO: 1.

8. The amphiphilic ligand conjugate, or pharmaceutically acceptable salt thereof, of claim 5, wherein the peptide comprises an amino acid sequence identical to SEQ ID NO: 2.

9. The amphiphilic ligand conjugate, or pharmaceutically acceptable salt thereof, of claim 5, wherein the peptide comprises an amino acid sequence identical to SEQ ID NO: 5.

10. The amphiphilic ligand conjugate, or pharmaceutically acceptable salt thereof, of claim 1, wherein the lipid of the amphiphilic ligand conjugate is a diacyl lipid.

11. The amphiphilic ligand conjugate, or a pharmaceutically acceptable salt thereof, of claim 10, wherein the diacyl lipid of the amphiphilic ligand conjugate comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.

12. The amphiphilic ligand conjugate, or pharmaceutically acceptable salt thereof of claim 11, wherein the lipid is 1,2-distearoyl-sn-glycero-3-phosphoethanolamine (DSPE).

13. The amphiphilic ligand conjugate, or pharmaceutically acceptable salt thereof, of claim 1, wherein the linker is present and is selected from the group consisting of a hydrophilic polymer, a string of hydrophilic amino acids, a polysaccharide, and an oligonucleotide, or a combination thereof.

14. The amphiphilic ligand conjugate, or pharmaceutically acceptable salt thereof, of claim 13, wherein the linker comprises “N” polyethylene glycol units, wherein N is between 24-50.

15. The amphiphilic ligand conjugate, or pharmaceutically acceptable salt thereof, of claim 14, wherein the linker comprises PEG24-amido-PEG24.

16. The amphiphilic ligand conjugate, or pharmaceutically acceptable salt thereof, of claim 1, wherein the amphiphilic ligand conjugate is trafficked to a lymph node.

17. The amphiphilic ligand conjugate, or pharmaceutically acceptable salt thereof, of claim 16, wherein the amphiphilic ligand conjugate is trafficked to an inguinal lymph node or an axillary lymph node.

18. The amphiphilic ligand conjugate, or pharmaceutically acceptable salt thereof, of claim 16, wherein the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

19. The amphiphilic ligand conjugate, or pharmaceutically acceptable salt thereof, of claim 1, wherein the lipid is conjugated directly or via a linker to the N-terminus of the EGFR peptide.

20. The amphiphilic ligand conjugate, or pharmaceutically acceptable salt thereof, of claim 1, wherein the lipid is conjugated directly or via a linker to the C-terminus of the EGFR peptide.

21. A method of stimulating an immune response to a target cell population or target tissue in a subject, the method comprising administering to the subject (1) an amphiphilic ligand conjugate, comprising a lipid, an EGFR peptide, and, optionally, a linker, and (2) an immune cell modified with an EGFR receptor, wherein the EGFR receptor binds the EGFR peptide of the amphiphilic ligand conjugate.

22. The method of claim 21, wherein the EGFR peptide comprises an amino acid sequence of SEQ ID NO: 1, 2, or 5.

23. The method of claim 21, further comprising administering an adjuvant to the subject.

24. The method of claim 23, wherein the adjuvant is an amphiphilic oligonucleotide conjugate comprising an immunostimulatory oligonucleotide conjugated to a lipid, with or without a linker.

25. The method of claim 21, wherein the EGFR peptide is a cyclic peptide.

26. The method of claim 21, wherein the peptide comprises an amino acid sequence identical to SEQ ID NO: 1.

27. The method of claim 21, wherein the peptide comprises an amino acid sequence identical to SEQ ID NO: 2.

28. The method of claim 21, wherein the peptide comprises an amino acid sequence identical to SEQ ID NO: 5.

29. The method of claim 21, wherein the EGFR receptor is an EGFRviii receptor.

30. The method of claim 21, wherein the lipid of the amphiphilic ligand conjugate inserts into a cell membrane under physiological conditions, binds albumin under physiological conditions, or both.

31. The method of claim 21, wherein the lipid of the amphiphilic ligand conjugate is a diacyl lipid.

32. The method of claim 31, wherein the diacyl lipid of the amphiphilic ligand conjugate comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.

33. The method of claim 32, wherein the lipid is 1,2-distearoyl-sn-glycero-3-phosphoethanolamine (DSPE).

34. The method of claim 21, wherein the linker is selected from the group consisting of a hydrophilic polymer, a string of hydrophilic amino acids, a polysaccharide, and an oligonucleotide, or a combination thereof.

35. The method of claim 34, wherein the linker comprises “N” polyethylene glycol units, wherein N is between 24-50.

36. The method of claim 35, wherein the linker comprises PEG24-amido-PEG24.

37. The method of claim 21, wherein the amphiphilic ligand conjugate is trafficked to a lymph node.

38. The method of claim 37, wherein the amphiphilic ligand conjugate is trafficked to an inguinal lymph node or an axillary lymph node.

39. The method of claim 37, wherein the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

40. The method of claim 21, wherein the immune cell is a T cell, a B cell, a natural killer (NK) cell, a macrophage, a neutrophil, a dendritic cell, a mast cell, an eosinophil, or a basophil.

41. The method of claim 40, wherein the immune cell is a T cell.

42. The method of claim 21, wherein the immune response is an anti-tumor immune response.

43. The method of claim 21, wherein the target cell population or the target tissue is a tumor cell population or a tumor tissue.

44. The method of claim 21, wherein the method comprises reducing or decreasing the size of the tumor tissue or inhibiting growth of the tumor cell population or the tumor tissue in the subject.

45. The method of claim 21, wherein the method comprises activating the immune cell, expanding the immune cell, and/or increasing proliferation of the immune cell.

46. The method of claim 21, wherein the subject has a disease, a disorder, or a condition associated with expression or elevated expression of the antigen.

47. The method of claim 21, wherein the subject is lymphodepleted prior to the administration of the amphiphilic lipid conjugate.

48. The method of claim 47, wherein the lymphodepletion is by sublethal irradiation.

49. The method of claim 21, wherein the subject is administered the amphiphilic ligand conjugate prior to receiving the immune cell modified with an EGFR receptor.

50. The method of claim 21, wherein the subject is administered the amphiphilic ligand conjugate after receiving the immune cell modified with an EGFR receptor.

51. The method of claim 21, wherein amphiphilic ligand conjugate of and the receiving immune cell modified with an EGFR receptor are administered simultaneously.

52. The method of claim 21, wherein the lipid is conjugated directly or via a linker to the N-terminus of the EGFR peptide.

53. The method of claim 21, wherein the lipid is conjugated directly or via a linker to the C-terminus of the EGFR peptide.

54. A kit comprising (1) an amphiphilic ligand conjugate or pharmaceutically acceptable salt thereof of any one of claims 1-20, and (2) an immune cell modified with an EGFR receptor, wherein the EGFR receptor binds the EGFR peptide of the amphiphilic ligand conjugate.

55. The kit of claim 54, wherein the EGFR receptor is an EGFRviii receptor.


SEQUENCE LISTING (12 sequences)

SEQ ID NO: 1 - protein, 19 aa, synthetic construct
  DISULFID 2..17: note = Intrachain disulfide bond
  REGION 19: note = C-terminal amphiphile conjugate
ACGADSYEMEEDGVRKCKC

SEQ ID NO: 2 - protein, 19 aa, synthetic construct
  DISULFID 3..18: note = Intrachain disulfide bond
  REGION 1: note = N-terminal amphiphile conjugate
CACGADSYEMEEDGVRKCK

SEQ ID NO: 3 - protein, 19 aa, synthetic construct
  DISULFID 2..17: note = Intrachain disulfide bond
  REGION 19: note = C-terminal N-methylsuccinimide
ACGADSYEMEEDGVRKCKC

SEQ ID NO: 4 - protein, 19 aa, synthetic construct
  DISULFID 3..18: note = Intrachain disulfide bond
  REGION 1: note = N-terminal N-methylsuccinimide
CACGADSYEMEEDGVRKCK

SEQ ID NO: 5 - protein, 18 aa, synthetic construct
ACGADSYEMEEDGVRKCK

SEQ ID NO: 6 - DNA, 25 bp, synthetic construct
  source 1..25: mol_type = other DNA
  misc_difference 1..5: note = This region may encompass 1-5 nucleotides
  misc_feature 1: note = 5' lipophilic compound attached
gggggtccatgacgttcctgacgtt

SEQ ID NO: 7 - DNA, 8345 bp, synthetic construct
  source 1..8345: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagccctcgagaagcttgccaccatggccctgcctgtgacagccctgctgctgcctctggctctgctgctgcatgccgctagacccgatgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgctggtggcggaggatctggcggaggcggatcttctggcggtggctctgatatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggaggcggcacaaagctggaaatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctccggaggcggcggagagggcagaggaagtcttctaacatgcggtgacgtggaggagaatcccggccctaggatggtctccaaaggggaggaagacaacatggctatcatcaaagaatttatgagatttaaggtccatatggaaggctcagtgaatggtcacgaattcgagatcgaaggcgaaggagagggtcggccttatgaggggacacagacagctaaacttaaggttacaaaaggtggaccattgccctttgcctgggacatcctcagtccacagtttatgtatgggagcaaagcctatgttaagcatcccgcagacatccctgattaccttaagctttcatttcccgaaggtttcaagtgggagagggtaatgaactttgaagacgggggggttgttaccgttacacaggatagcagtcttcaagatggcgaatttatctataaggtgaaactgcggggcaccaactttcccagcgatgggcctgtgatgcaaaagaaaacaatgggatgggaagcaagtagcgaaaggatgtacccagaagatggtgcccttaagggagagatcaagcaacggcttaaactgaaagatggtggacactacgatgcagaagtcaaaacaacttacaaagccaaaaagcccgttcaactcccaggtgcctacaacgttaatattaagctggacattacaagtcataatgaagattacactatagttgagcagtacgagcgggccgagggcaggcacagcacaggtggtatggacgaactgtataagtaacgctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtcgacggtaccgcgggcccgggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 8 - DNA, 7757 bp, synthetic construct
  source 1..7757: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagccctcgagaagcttgccaccatggcattgcccgtgaccgccctgctgctgccactggccttgttgctccacgccgcgcggccagaacagaagctgatcagcgaggaggatctggatgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgctggtggcggaggatctggcggaggcggatcttctggcggtggctctgatatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggaggcggcacaaagctggaaatcaagcggatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagatccggaggcggcggagagggcagaggaagtcttctaacatgcggtgacgtggaggagaatcccggccctaggatggtctccaaaggggaggaagacaacatggctatcatcaaagaatttatgagatttaaggtccatatggaaggctcagtgaatggtcacgaattcgagatcgaaggcgaaggagagggtcggccttatgaggggacacagacagctaaacttaaggttacaaaaggtggaccattgccctttgcctgggacatcctcagtccacagtttatgtatgggagcaaagcctatgttaagcatcccgcagacatccctgattaccttaagctttcatttcccgaaggtttcaagtgggagagggtaatgaactttgaagacgggggggttgttaccgttacacaggatagcagtcttcaagatggcgaatttatctataaggtgaaactgcggggcaccaactttcccagcgatgggcctgtgatgcaaaagaaaacaatgggatgggaagcaagtagcgaaaggatgtacccagaagatggtgcccttaagggagagatcaagcaacggcttaaactgaaagatggtggacactacgatgcagaagtcaaaacaacttacaaagccaaaaagcccgttcaactcccaggtgcctacaacgttaatattaagctggacattacaagtcataatgaagattacactatagttgagcagtacgagcgggccgagggcaggcacagcacaggtggtatggacgaactgtataagtaagtcgacggtaccgcgggcccgggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 9 - DNA, 8321 bp, synthetic construct
  source 1..8321: mol_type = other DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagccctcgagaagcttgccaccatgggtgtccctacccagctcctgggactgctcctgctgtggatcaccgacgccatctgcgaggtgcaggtgttggagtctgggggaggcttggtacagcctggggggtccctgagactctcctgtgcagcctctggattcacctttagcagctatgccatgagctgggtccgccaggctccagggaaggggctggagtgggtctcggctattagtggtagtggtggtagtacaaactacgcagactccgtgaagggccggttcaccatctccagagacaattccaagaacacactgtatctgcaaatgaacagcctgagagccgaggacacggccgtctattactgtgctgggagcagtggctggtccgagtactggggccagggaaccctggtcaccgtctcctcgggtggaggtggatcaggtggaggtggatctggtggaggtggatctgacatccagatgacccagtctccatcctccctgtctgcatctgtaggagacagagtcaccatcacttgccgggctagtcagggcattagaaataatttagcctggtatcagcagaaaccagggaaagcccctaagcgcctgatctatgctgcctccaatttgcaaagtggggtcccatcaaggttcaccggcagtggatctgggacagaattcactctcatagtcagcagcctgcagcctgaagattttgcgacttattactgtctacagcatcacagttacccgctcacttccggcggagggaccaaggtggagatcaaaatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagatccggaggcggcggagagggcagaggaagtcttctaacatgcggtgacgtggaggagaatcccggccctaggatggtctccaaaggggaggaagacaacatggctatcatcaaagaatttatgagatttaaggtccatatggaaggctcagtgaatggtcacgaattcgagatcgaaggcgaaggagagggtcggccttatgaggggacacagacagctaaacttaaggttacaaaaggtggaccattgccctttgcctgggacatcctcagtccacagtttatgtatgggagcaaagcctatgttaagcatcccgcagacatccctgattaccttaagctttcatttcccgaaggtttcaagtgggagagggtaatgaactttgaagacgggggggttgttaccgttacacaggatagcagtcttcaagatggcgaatttatctataaggtgaaactgcggggcaccaactttcccagcgatgggcctgtgatgcaaaagaaaacaatgggatgggaagcaagtagcgaaaggatgtacccagaagatggtgcccttaagggagagatcaagcaacggcttaaactgaaagatggtggacactacgatgcagaagtcaaaacaacttacaaagccaaaaagcccgttcaactcccaggtgcctacaacgttaatattaagctggacattacaagtcataatgaagattacactatagttgagcagtacgagcgggccgagggcaggcacagcacaggtggtatggacgaactgtataagtaacgctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtcgacggtaccgcgggcccgggatccgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatccatcgatggagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 10 - DNA, 10221 bp, synthetic construct
  source 1..10221: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgaggttctgagagatctaccgccgccaccatgcgaccctcagggaccgcgagaaccacactgctggtgttgctgaccgcgctctgcgccgcaggtggggcgttggaggaaaagaaaggaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcaggggaccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggccaaagataccatctattgccactgggattgtgggtggcctcctcttcatagtggtggtggcccttgggattggcctattcatgcgaagacgtcacattgttcgaaagcgtacactacgccgcctgcttcaagagagagagctcgtggaacctctcacacccagcggagaagctccaaaccaagcccacttgaggatattaaaggaaacagaattcaaaaagatcaaagttctgggttcgggagcatttggcacagtgtataagggtctctggatcccagaaggtgagaaagtaaaaatcccggtggccatcaaggagttaagagaagccacatctccaaaagccaacaaagaaatccttgacgaagcctatgtgatggctagtgtggacaaccctcatgtatgccgcctcctgggcatctgtctgacctccactgtccagctcattacacagctcatgccctacggttgcctcctggactacgtccgagaacacaaggacaacattggctcccagtacctcctcaactggtgtgtgcagattgcaaagggcatgaactacctggaagatcggcgtttggtgcaccgtgacttggcagccaggaatgtactggtgaagacaccacagcatgtcaagatcacagattttgggctggccaaactgcttggtgctgaagagaaagaatatcatgccgaggggggcaaagtgcctatcaagtggatggctttggaatcaattttacaccgaatttatacacaccaaagtgatgtctggagctatggtgtcactgtgtgggaactgatgacctttgggtccaagccttatgatggaatcccagcaagtgacatctcatccatcctagagaaaggagagcgccttccacagccaccttccggaggcggcggagagggcagaggaagtcttctaacatgcggtgacgtggaggagaatcccggccctaggatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagccaccaacttcagcctgctgaagcaggccggcgacgtggaggagaaccctggccctcctaggatggtgaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgcgtgggcactcagctgattcctggtgtcaccgtcttggtctacttgcctttcttccatgctttcggctttagcattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagcgctaacattcactctctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggctggttgcattctggtgattttggatattacgacgaagatgagcatttttacgtcgtggatcgttacaaggagctgatcaaatacaagggtagccaggttgctccagctgagttggaggagattctgttgaaaaatccatgcattcgcgatgtcgctgtggtcggcattcctgatctggaggccggcgaactgccttctgctttcgttgtcaagcagcctggtaaagaaattaccgccaaagaagtgtatgattacctggctgaacgtgtgagccatactaagtacttgcgtggcggcgtgcgttttgttgactccatccctcgtaacgtaacaggcaaaattacccgcaaggagctgttgaaacaattgttggagaaggccggcggttaacgctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagctcagaacgaattcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 11 - protein, 1136 aa, Homo sapiens
DLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 12 - DNA, 24 bp, synthetic construct
  source 1..24: mol_type = other DNA
tcgtcgttttgtcgttttgtcgtt